(19) United States
(12) Patent Application Publication
(10) Pub. No.: US 2007/0027199 A1
(43) Pub. Date: Feb. 1, 2007

(54) Cycloalkyl amino-hydantoin compounds and use thereof for beta-secretase modulation

(75) Inventors: Michael Sotirios Malamas, Jamison, PA (US); Iwan Suwandi Gunawan, Somerset, NJ (US); James Joseph Erdei, Philadelphia, PA (US); Pawel Wojciech Nowak, Montvale, NJ (US); Joseph Raymond Stock, Monroe, NY (US); Yinfa Yan, Bedminster, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/495,261

(22) Filed: Jul. 28, 2006

Related U.S. Application Data
(60) Provisional application No. 60/704,867, filed on Jul. 29, 2005.

(51) Int. Cl. A61K 31/4178; A61K 31/4166; C07D 403/02

(52) U.S. Cl. 514/388; 548/321.5; 548/311.1

(57) ABSTRACT

The present invention provides a 2-amino-5-cycloalkyl-hydantoin compound of formula I 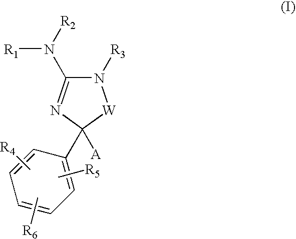 The present invention also provides methods and compositions for the inhibition of β-secretase (BACE) and the treatment of β-amyloid deposits and neurofibrillary tangles.

This application claims the benefit under 35 U.S.C. §119(e) to co-pending U.S. Provisional Application No. 60/704,867, filed Jul. 29, 2005, which is hereby incorporated by reference in its entirety.

### BACKGROUND OF THE INVENTION

β-amyloid deposits and neurofibrillary tangles are two major pathologic characterizations associated with Alzheimer's disease (AD). Clinically, AD is characterized by the of loss of memory, cognition, reasoning, judgment, and orientation. Also affected, as the disease progresses, are motor, sensory, and linguistic abilities until global impairment of multiple cognitive functions occurs. These cognitive losses take place gradually, but typically lead to severe impairment and eventual death in 4-12 years.

Amyloidogenic plaques and vascular amyloid angiopathy also characterize the brains of patients with Trisomy 21 (Down's Syndrome), Hereditary Cerebral Hemorrhage with Amyloidosis of the Dutch-type (HCHWA-D), and other neurodegenerative disorders. Neurofibrillary tangles also occur in other neurodegenerative disorders including dementia-inducing disorders (Varghese, J., et al, Journal of Medicinal Chemistry, 2003, 46, 4625-4630).

β-amyloid deposits are predominately an aggregate of Aβ peptide, which in turn is a product of the proteolysis of amyloid precursor protein (APP). More specifically, Aβ peptide results from the cleavage of APP at the C-terminus by one or more γ-secretases, and at the N-terminus by β-secretase enzyme (BACE), also known as aspartyl protease, as part of the β-amyloidogenic pathway.

BACE activity is correlated directly to the generation of Aβ peptide from APP (Sinha, et al, Nature, 1999, 402, 537-540), and studies increasingly indicate that the inhibition of BACE inhibits the production of Aβ peptide (Roberds, S. L., et al, Human Molecular Genetics, 2001, 10, 1317-1324).

Therefore, it is an object of this invention to provide compounds which are inhibitors of β-secretase and are useful as therapeutic agents in the treatment, prevention or amelioration of a disease or disorder characterized by elevated β-amyloid deposits or β-amyloid levels in a patient.

It is another object of this invention to provide therapeutic methods and pharmaceutical compositions useful for the treatment, prevention or amelioration of a disease or disorder characterized by elevated β-amyloid deposits or β-amyloid levels in a patient.

It is a feature of this invention that the compounds provided may also be useful to further study and elucidate the β-secretase enzyme.

These and other objects and features of the invention will become more apparent by the detailed description set forth hereinbelow.

### SUMMARY OF THE INVENTION

The present invention provides a compound of formula I 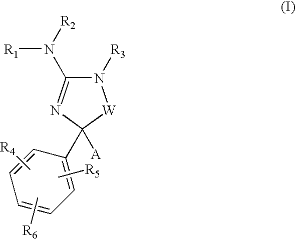
wherein
A is cycloalkyl;
W is CO, CS or CH₂;
R₁, R₂, and R₃ are each independently H, or an alkyl, cycloalkyl, cycloheteroalkyl, aryl or heteroaryl group, each group optionally substituted, or R₁ and R₂ may be taken together with the atom to which they are attached to form an optionally substituted 5- to 7-membered ring optionally interrupted by an additional heteroatom selected from O, N or S;
R₄, R₅, and R₆ are each independently H, halogen, NO₂, CN, OR₇, COR₇, CO₂R₇, CONR₈R₉, NR₈R₉, NR₈COR₇, NR₈SO₂R₁₀, SO₂NR₈R₉ or SOₙR₁₀ or an alkyl, alkenyl, alkynyl, cycloalkyl, cycloheteroalkyl, aryl, or heteroaryl group, each group optionally substituted, or when attached to adjacent carbon atoms R₄ and R₅ or R₅ and R₆ may be taken together with the atoms to which they are attached to form a an optionally substituted 5- to 7-membered ring optionally interrupted by one, two or three heteroatoms selected from O, N or S;
n is 0, 1, or 2;
R₇ is independently at each occurrence H, or an alkyl, alkenyl, alkynyl, cycloalkyl, cycloheteroalkyl, aryl, or heteroaryl group each group optionally substituted;

R₈ and R₉ are each independently at each occurrence H, OR₇, COR₇, CO₂R₇ or an alkyl, alkenyl, alkynyl, cycloalkyl, cycloheteroalkyl, aryl, or heteroaryl group, each group optionally substituted, or R₈ and R₉ may be taken together with the atom to which they are attached to form an optionally substituted 5- to 7-membered ring optionally interrupted by an additional heteroatom selected from O, N or S; and
R₁₀ is independently at each occurrence an alkyl, alkenyl, alkynyl, cycloalkyl, cycloheteroalkyl, aryl or heteroaryl group each group optionally substituted; or
   a tautomer thereof, a stereoisomer thereof or a pharmaceutically acceptable salt thereof.

The present invention also relates to the use of cycloalkyl amino-hydantoin compounds for the treatment of β-amyloid deposits and neurofibrillary tangles. These compounds are particularly useful in treating Alzheimer's disease, cognitive impairment, Down's Syndrome, HCHWA-D, cognitive decline, senile dementia, cerebral amyloid angiopathy, degenerative dementia, or other neurodegenerative disorders.

### DETAILED DESCRIPTION OF THE INVENTION

Alzheimer's disease (AD) is a major degenerative disease of the brain which presents clinically by progressive loss of memory, cognition, reasoning, judgement and emotional stability and gradually leads to profound mental deteoriation and death. The exact cause of AD is unknown, but increasing evidence indicates that amyloid beta peptide (A-beta) plays a central role in the pathogenesis of the disease. (D. B. Schenk; R. E. Rydel et al, Journal of Medicinal Chemistry, 1995, 21, 4141 and D. J. Selkoe, Physiology Review, 2001, 81, 741). Patients with AD exhibit characteristic neuropathological markers such as neuritic plaques (and in β-amyloid angiopathy, deposits in cerebral blood vessels) as well as neurofibrillary tangles detected in the brain at autopsy. A-beta is a major component of neuritic plaques in AD brains. In addition, β-amyloid deposits and vascular β-amyloid angiopathy also characterize individuals with Downs Syndrome, Hereditary Cerebral Hemmorhage with Amyloidosis of the Dutch type and other neurodegenreative and dementia-inducing disorders. Over expression of the amyloid precursor protein (APP), altered cleavage of APP to A-beta or a decrease in the clearance of A-beta from a patient's brain may increase the levels of soluble or fibrullar forms of A-beta in the brain. The β-site APP cleaving enzyme, BACE1, also called memapsin-2 or Asp-2, was identified in 1999 (R. Vassar, B. D. Bennett, et al, Nature, 1999, 402, 537). BACE1 is a membrane-bound aspartic protease with all the known functional properties and characteristics of β-secretase. Low molecular weight, non-peptide, non-substrate-related inhibitors of BACE1 or β-secretase are earnestly sought both as an aid in the study of the β-secretase enzyme and as potential therapeutic agents.

Surprisingly, it has now been found that 2-amino-5-cycloalkyl-hydantoin compounds of formula I demonstrate inhibition of β-secretase and the selective inhibition of BACE1. Advantageously, said cycloalkyl-hydantoin compounds may be used as effective therapeutic agents for the treatment, prevention or amelioration of a disease or disorder characterized by elevated β-amyloid deposits or β-amyloid levels in a patient. Accordingly, the present invention provides a 2-amino-5-cycloalkyl-hydantoin compound of formula I 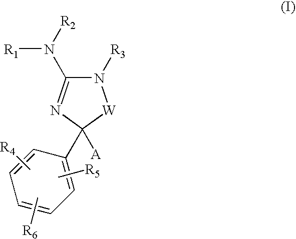
wherein
A is cycloalkyl;
W is CO, CS or CH₂;
R₁, R₂, and R₃ are each independently H, or an alkyl, cycloalkyl, cycloheteroalkyl, aryl or heteroaryl group, each group optionally substituted, or R₁ and R₂ may be taken together with the atom to which they are attached to form an optionally substituted 5- to 7-membered ring optionally interrupted by an additional heteroatom selected from O, N or S;
R₄, R₅, and R₆ are each independently H, halogen, NO₂, CN, OR₇, COR₇, CO₂R₇, CONR₈R₉, NR₈R₉, NR₈COR₇, NR₈SO₂R₁₀, SO₂NR₈R₉ or SOₙR₁₀ or an alkyl, alkenyl, alkynyl, cycloalkyl, cycloheteroalkyl, aryl, or heteroaryl group, each group optionally substituted, or when attached to adjacent carbon atoms R₄ and R₅ or R₅ and R₆ may be taken together with the atoms to which they are attached to form a an optionally substituted 5- to 7-membered ring optionally interrupted by one, two or three heteroatoms selected from O, N or S;
n is 0, 1, or 2;
R₇ is independently at each occurrence H, or an alkyl, alkenyl, alkynyl, cycloalkyl, cycloheteroalkyl, aryl, or heteroaryl group each group optionally substituted;
R₈ and R₉ are each independently at each occurrence H, OR₇, COR₇, CO₂R₇ or an alkyl, alkenyl, alkynyl, cycloalkyl, cycloheteroalkyl, aryl, or heteroaryl group, each group optionally substituted, or R₈ and R₉ may be taken together with the atom to which they are attached to form an optionally substituted 5- to 7-membered ring optionally interrupted by an additional heteroatom selected from O, N or S; and
R₁₀ is independently at each occurrence an alkyl, alkenyl, alkynyl, cycloalkyl, cycloheteroalkyl, aryl or heteroaryl group each group optionally substituted; or
   a tautomer thereof, a stereoisomer thereof or a pharmaceutically acceptable salt thereof.

It is understood that the claims encompass all possible stereoisomers, tautomers, and prodrugs. Moreover, unless stated otherwise, each alkyl, alkenyl, alkynyl, cycloalkyl, cycloheteroalkyl, aryl, or heteroaryl is contemplated as being optionally substituted.

A may be monocyclic cycloalkyl or polycyclic cycloalkyl.

In one embodiment, A is polycyclic.

In a preferred embodiment, A is a bridged polycyclic cycloalkyl group, such as norbornyl or adamantyl. Thus, preferred A constituents are those of Formula II or III: 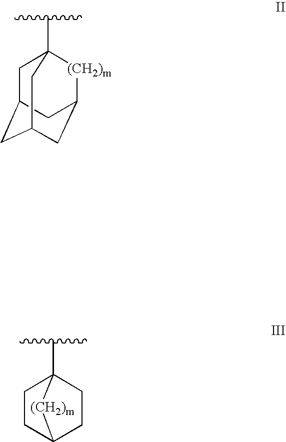
wherein m is 1 or 2. More preferably, A is adamantyl.

In other embodiments, A is a monocyclic cycloalkyl group.

In certain embodiments R₅ is alkyl, alkoxy or haloalkoxy. Preferred haloalkoxy groups are OCF₃ and OCHF₂.

The term “cycloalkyl” as used in the specification and claims designates cyclized alkyl chains having the specified number of carbon atoms, e.g., cyclopropyl, cyclobutyl, cyclopentyl, and cyclohexyl, up to 20 carbon atoms, which may be a single ring (monocyclic) or multiple rings (polycyclic, including spiro, fused, and bridged rings, up to three rings) fused together or linked covalently.

The term “cycloheteroalkyl” designates a five- to seven-membered cycloalkyl ring system containing 1 or 2 heteroatoms, which may be the same or different, selected from N, O, or S and optionally containing one double bond. Exemplary cycloheteroalkyl ring systems are the following rings wherein X₁ is NR, O or S; and R is H or an optional substituent as described below:

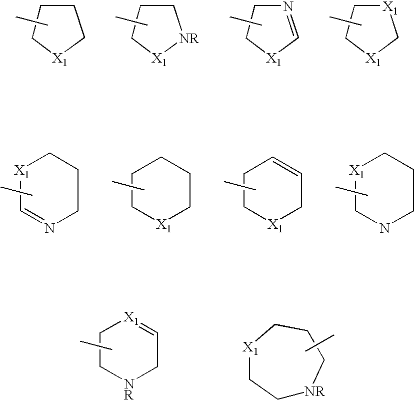

As used herein, the term “alkyl” includes both branched and straight-chain saturated aliphatic hydrocarbon groups having the specified number of carbon atoms, e.g. methyl, ethyl, propyl, isopropyl, isobutyl, secondary butyl, tertiary butyl, isopentyl, neopentyl, isohexyl or the like. The term “alkyl” further includes both unsubstituted and mono-, di- and tri-substituted hydrocarbon groups, with halogen substitution particularly preferred.

The term “alkenyl” refers to an unsaturated or partially unsaturated aliphatic hydrocarbon group having the specified number of carbon atoms, for example ethenyl, 1-propenyl, 2, butenyl, etc. The term “alkenyl” further includes both unsubstituted and mono-, di- and tri-substituted hydrocarbon groups, with halogen substitution particularly preferred.

The term “alkynyl” refers to an alkyl group having one or more triple carbon-carbon bonds. Alkynyl groups preferably contain 2 to 6 carbon atoms. Examples of alkynyl groups include, but are not limited to, ethynyl, propynyl, butynyl, pentynyl, and the like. In some embodiments, alkynyl groups can be substituted with up to four substituent groups, as described below.

The term “halogen” designates fluorine, chlorine, iodine, and bromine.

The term “aryl” designates an aromatic carbocyclic moiety of up to 20 carbon atoms, which may be a single ring (monocyclic) or multiple rings (polycyclic, up to three rings) fused together or linked covalently. Examples of aryl moieties include, but are not limited to, phenyl, 1-naphthyl, 2-naphthyl, dihydronaphthyl, tetrahydronaphthyl, biphenyl. anthryl, phenanthryl, fluorenyl, indanyl, biphenylenyl, acenaphthenyl, acenaphthylenyl, or the like.

The term “heteroaryl” designates an aromatic 5-membered to 7-membered carbon-containing ring incorporating at least one nitrogen, oxygen or sulfur atom. Such heteroaryl ring systems include pyrrolyl, pyrazolyl, imidazolyl, pyridinyl, primidinyl, pyrazinyl, azolyl, oxazolyl, thiazolyl, imidazolyl, furyl, thienyl, quinolinyl, isoquinolinyl, indolyl, benzothienyl, benzofuranyl, benzisoxazolyl or the like.

An optionally substituted moiety may be substituted with one or more substituents. The substituent groups which are optionally present may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl, lower alkoxy or haloalkoxy groups. Typically, 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms.

Optional substituents may be, for example, alkyl, e.g. methyl or ethyl, alkoxy, e.g. methoxy, haloalkoxy, e.g. trifluoromethoxy or difluoromethoxy, halogen, aryloxy, e.g. phenoxy, haloalkyl, e.g. trifluoromethyl, heteroaryl, e.g. furyl, cycloalkyl, e.g. cyclopentyl or cyclohexyl, carbamoyl, carboxyl, alkoxycarbonyl or the like, preferably halogen atoms or lower alkyl, lower alkoxy or haloalkoxy groups, wherein ‘lower’ denotes 1 to 4 carbon atoms. Typically, 0-4 substituents may be present.

The compounds of the present invention can be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Compounds of the invention may exist as one or more tautomers. One skilled in the art will recognize that compounds of formula I may also exist as the tautomer It as shown below.

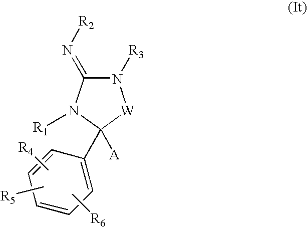

Tautomers often exist in equilibrium with each other. As these tautomers interconvert under environmental and physiological conditions, they provide the same useful biological effects. The present invention includes mixtures of such tautomers as well as the individual tautomers of Formula I and Formula It.

The compounds of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, preferably less than about 50%, more preferably less than about 75%, and even more preferably less than about 90%.

Preferred compounds of the invention are those compounds of formula I wherein W is CO. Also preferred are those compounds of formula I wherein R₁ and R₂ are each independently H or alkyl. Another group of preferred compounds are those compounds of formula I wherein R₅ is OR₇. A further group of preferred compounds are those formula I compounds wherein R₃ is alkyl.

More preferred compounds of the invention are those compounds of formula I wherein W is CO and A is adamantyl. Another group of more preferred compounds is those compounds of formula I wherein W is CO; A is adamantyl and R₁ and R₂ are H. A further group of more preferred compounds are those compounds of formula I wherein W is CO; A is adamantyl and R₅ is difluoromethoxy.

Preferred compounds of Formula I include:
(5S)-5-(1-adamantyl)-2-amino-5-[4-(difluoromethoxy)phenyl-3-methyl-3,5-dihydro-4H-imidazol-4-one;
2-amino-5-bicyclo[2.2.1]hept-1-yl-5-(4-methoxy-3-methylphenyl)-3-methyl-3,5-dihydro-4H-imidazol-4-one;
5-(1-adamantyl)-2-amino-5-(4-methoxyphenyl)-3-methyl-3,5-dihydro-4H-imidazol-4-one;
5-(1-adamantyl)-2-amino-5-(4-ethoxyphenyl)-3-methyl-3,5-dihydro-4H-imidazo-4-one;
5-(1-adamantyl)-2-amino-5-(4-butoxyphenyl)-3-methyl-3,5-dihydro-4H-imidazol-4-one;
5-(1-adamantyl)-2-amino-5-(3-ethyl-4-methoxyphenyl)-3-methyl-3,5-dihydro-4H-imidazol-4-one;
5-(1-adamantyl)-2-amino-5-(4-methoxy-3,5-dimethylphenyl)-3-methyl-3,5-dihydro-4H-imidazol-4-one;
5-(1-adamantyl)-2-amino-3-methyl-5-phenyl-3,5-dihydro-4H-imidazol-4-one;
5-(1-adamantyl)-2-amino-5-(4-methoxy-3-methylphenyl)-3-methyl-3,5-dihydro-4H-imidazol -4-one;
(5S)-5-(1-adamantyl)-2-amino-5-(4-methoxy-3-methylphenyl)-3-methyl-3,5-dihydro -4H-imidazol-4-one;
(5R)-5-(1-adamantyl)-2-amino-5-(4-methoxy-3-methylphenyl)-3-methyl-3,5-dihydro -4H-imidazol-4-one;
5-(1-adamantyl)-2-amino-5-(3,4-dimethoxyphenyl)-3-methyl-3,5-dihydro-4H-imidazol -4-one;
5-(1-adamantyl)-2-amino-5-(4-methoxy-2,3-dimethylphenyl)-3-methyl-3,5-dihydro-4H-imidazol -4-one;
2-amino-5-bicyclo[2.2.1]hept-2-yl-5-(4-methoxy-3-methylphenyl)-3-methyl-3,5-dihydro-4H-imidazol-4-one;
2-amino-5-hexahydro-2,5-methanopentalen-3a(1H)-yl-5-(4-methoxy-3-methylphenyl) -3-methyl-3,5-dihydro-4H-imidazol-4-one;
5-(1-adamantyl)-2-amino-3-methyl-5-(4′-methyl-1,1′-biphenyl-3-yl)-3,5-dihydro-4H-imidazol -4-one;
5-(1-adamantyl)-2-amino-5-(4′-methoxy-1,1′-biphenyl-3-yl)-3-methyl-3,5-dihydro-4H-imidazol -4-one;
5-(1-adamantyl)-2-amino-3-methyl-5-(3′-methyl-1,1′-biphenyl-3-yl)-3,5-dihydro-4H-imidazol -4-one;
5-(1-adamantyl)-2-amino-5-(3′-methoxy-1,1′-biphenyl-3-yl)-3-methyl-3,5-dihydro-4H-imidazol -4-one;
5-(1-adamantyl)-2-amino-5-(3′,4′-dimethyl-1,1′-biphenyl-3-yl)-3-methyl-3,5-dihydro -4H-imidazol-4-one;
3′-[4-(1-adamantyl)-2-amino-1-methyl-5-oxo-4,5-dihydro-1H-imidazol-4-yl]-1,1′-biphenyl-3-carbonitrile;
5-(1-adamantyl)-2-amino-5-[3-(3-furyl)phenyl]-3-methyl-3,5-dihydro-4H-imidazo-4-one;
3′-[4-(1-adamantyl)-2-amino-1-methyl-5-oxo-4,5-dihydro-1H-imidazol-4-yl]-1,1′-biphenyl-4-carbonitrile;
3′-[4-(1-adamantyl)-2-amino-1-methyl-5-oxo-4,5-dihydro-1H-imidazol-4-yl]-1,1′-biphenyl-4-carbonitrile;
5-(1-adamantyl)-2-amino-5-(3′,4′-difluoro-1,1′-biphenyl-3-yl)-3-methyl-3,5-dihydro-4H-imidazol -4-one;
5-(1-adamantyl)-2-amino-5-(1,1′-biphenyl-3-yl)-3-methyl-3,5-dihydro-4H-imidazol-4-one;
5-(1-adamantyl)-2-amino-3-methyl-5-(2′-methyl-1,1′-biphenyl-3-yl)-3,5-dihydro-4H-imidazol -4-one;
2-amino-5-cyclohexyl-3-(3,5-difluorobenzyl)-5-phenyl-3,5-dihydro-4H-imidazol-4-one;
5-cyclohexyl-3-ethyl-2-imino-5-phenylimidazolidin-4-one;
5-cyclohexyl-2-imino-5-phenyl-3-propylimidazolidin-4-one;
5-cyclohexyl-3-(3-hydroxypropyl)-2-imino-5-phenylimidazolidin-4-one;
2-amino-5-cyclohexyl-3-(2,2-diethoxyethyl)-5-phenyl-3,5-dihydro-4H-imidazo-4-one;
2-amino-5-cyclohexyl-5-phenyl-3-(2-phenylethyl)-3,5-dihydro-4H-imidazol-4-one;
5-cyclohexyl-2-imino-3-methyl-5-phenylimidazolidin-4-one;
2-amino-5-cyclohexyl-5-phenyl-3-(tetrahydrofuran-2-ylmethyl)-3,5-dihydro-4H-imidazol -4-one;
2-amino-5-cyclohexyl-3-(2-fluoroethyl)-5-phenyl-3,5-dihydro-4H-imidazol-4-one;
2-amino-5-cyclohexyl-3-[2-(difluoromethoxy)benzyl]-5-phenyl-3,5-dihydro-4H-imidazol -4-one;
N-[(2-amino-4-cyclohexyl-5-oxo-4-phenyl-4,5-dihydro-1H-imidazol-1-yl)acetyl]-L-aspartic acid;
N-[(2-amino-4-cyclohexyl-5-oxo-4-phenyl-4,5-dihydro-1H-imidazol-1-yl)acetyl]-D-aspartic acid;
trans-4-[(2-amino-4-cyclohexyl-5-oxo-4-phenyl-4,5-dihydro-1H-imidazol-1-yl)methyl]cyclohexanecarboxylic acid;
6-(2-amino-4-cyclohexyl-5-oxo-4-phenyl-4,5-dihydro-1H-imidazol-1-yl)hexanoic acid;
5-(2-amino-4-cyclohexyl-5-oxo-4-phenyl-4,5-dihydro-1H-imidazol-1-yl)pentanoic acid;
4-(2-amino-4-cyclohexyl-5-oxo-4-phenyl-4,5-dihydro-1H-imidazol-1-yl)butanoic acid;
2-amino-5-cyclohexyl-3-(5-hydroxypentyl)-5-phenyl-3,5-dihydro-4H-imidazol-4-one;
3-(2-amino-4-cyclohexyl-5-oxo-4-phenyl-4,5-dihydro-1H-imidazol-1-yl)propanoic acid;
3-(2-amino-4-cyclohexyl-5-oxo-4-phenyl-4,5-dihydro-1H-imidazol-1-yl)-2-methylpropanoic acid;
2-amino-3-benzyl-5-cyclohexyl-5-phenyl-3,5-dihydro-4H-imidazol-4-one;
2-amino-5-cyclohexyl-3-isobutyl-5-phenyl-3,5-dihydro-4H-imidazol-4-one;
2-amino-5-cyclohexyl-3-hexyl-5-phenyl-3,5-dihydro-4H-imidazol-4-one;
2-amino-3,5-dicyclohexyl-5-phenyl-3,5-dihydro-4H-imidazol-4-one;
2-amino-5-cyclohexyl-3-(4-hydroxybutyl)-5-phenyl-3,5-dihydro-4H-imidazol-4-one;
(2-amino-4-cyclohexyl-5-oxo-4-phenyl-4,5-dihydro-1H-imidazol-1-yl)acetic acid;
2-amino-5-cyclohexyl-3-(cyclohexylmethyl)-5-phenyl-3,5-dihydro-4H-imidazol-4-one;
2-amino-5-cyclohexyl-3-(2-furylmethyl)-5-phenyl-3,5-dihydro-4H-imidazol-4-one;
2-amino-5-cyclohexyl-3-(4-hydroxyphenyl)-5-phenyl-3,5-dihydro-4H-imidazol-4-one;
2-amino-5-cyclohexyl-3-(3-hydroxyphenyl)-5-phenyl-3,5-dihydro-4H-imidazol-4-one;
2-amino-5-cyclohexyl-5-phenyl-3-(thien-2-ylmethyl)-3,5-dihydro-4H-imidazol-4-one;
2-amino-5-cyclohexyl-3-(4-methoxyphenyl)-5-phenyl-3,5-dihydro-4H-imidazol-4-one;
2-amino-5-cyclohexyl-5-phenyl-3-(2-thien-2-ylethyl)-3,5-dihydro-4H-imidazol-4-one;
2-amino-5-cyclohexyl-3-[2-(4-hydroxyphenyl)ethyl]-5-phenyl-3,5-dihydro-4H-imidazol -4-one;
[4-(2-amino-4-cyclohexyl-5-oxo-4-phenyl-4,5-dihydro-1H-imidazol-1-yl)phenyl]acetic acid;
4-[(2-amino-4-cyclohexyl-5-oxo-4-phenyl-4,5-dihydro-1H-imidazol-1-yl)methyl]benzoic acid;
5-(2-amino-4-cyclohexyl-5-oxo-4-phenyl-4,5-dihydro-1H-imidazol-1-yl)-2-hydroxybenzoic acid;
ethyl 3-(2-amino-4-cyclohexyl-5-oxo-4-phenyl-4,5-dihydro-1H-imidazol-1-yl)benzoate;
5-cyclobutyl-2-imino-3-methyl-5-phenylimidazolidin-4-one;
5-(2-adamantyl)-2-imino-3-methyl-5-phenylimidazolidin-4-one;
5-cyclopentyl-2-imino-3-methyl-5-phenylimidazolidin-4-one;
5-cyclobutyl-3-ethyl-2-imino-5-phenylimidazolidin-4-one;
5-cycloheptyl-3-ethyl-2-imino-5-phenylimidazolidin-4-one;
5-(2-adamantyl)-3-ethyl-2-imino-5-phenylimidazolidin-4-one;
5-cyclopentyl-3-ethyl-2-imino-5-phenylimidazolidin-4-one;
5-cyclobutyl-2-imino-5-phenyl-3-propylimidazolidin-4-one;
5-cycloheptyl-2-imino-5-phenyl-3-propylimidazolidin-4-one;
5-(2-adamantyl)-2-imino-5-phenyl-3-propylimidazolidin-4-one;
5-cyclopentyl-2-imino-5-phenyl-3-propylimidazolidin-4-one;
5-cyclobutyl-3-(3-hydroxypropyl)-2-imino-5-phenyl-3-propylimidazolidin-4-one;
5-cycloheptyl-3-(3-hydroxypropyl)-2-imino-5-phenylimidazolidin-4-one;
5-(2-adamantyl)-3-(3-hydroxypropyl)-2-imino-5-phenylimidazolidin-4-one;
5-cyclopentyl-3-(3-hydroxypropyl)-2-imino-5-phenylimidazolidin-4-one;
5-cyclohexyl-2-imino-3-methyl-5-(2-methylphenyl)imidazolidin-4-one;
5-(3-benzylphenyl)-5-cyclohexyl-2-imino-3-methylimidazolidin-4-one;
5-cyclohexyl-2-imino-3-methyl-5-(3-methylphenyl)imidazolidin-4-one;
5-cyclohexyl-2-imino-3-methyl-5-(4-methylphenyl)imidazolidin-4-one;
5-cyclohexyl-5-(4-fluorophenyl)-2-imino-3-methyl imidazolidin-4-one;
5-cyclohexyl-2-imino-5-(3-methoxyphenyl)-3-methyl imidazolidin-4-one;
5-cyclohexyl-5-(3,4-dichlorophenyl)-2-imino-3-methylimidazolidin-4-one;
5-cyclohexyl-2-imino-3-methyl-5-(4-phenoxyphenyl)imidazolidin-4-one;
5-(3-chlorophenyl)-5-cyclohexyl-2-imino-3-methylimidazolidin-4-one;
5-cyclohexyl-5-(3,5-dichlorophenyl)-2-imino-3-methylimidazolidin-4-one;
5-(1,1′-biphenyl-2-yl)-5-cyclohexyl-2-imino-3-methylimidazolidin-4-one;
5-(1,1′-biphenyl-4-yl)-5-cyclohexyl-2-imino-3-methylimidazolidin-4-one;
5-cyclohexyl-5-(2,5-dimethylphenyl)-2-imino-3-methylimidazolidin-4-one;
5-cyclohexyl-2-imino-3-methyl-5-[4-(trifluoromethyl)phenyl]imidazolidin-4-one;
5-cyclohexyl-2-imino-5-(2-methoxyphenyl)-3-methylimidazolidin-4-one;
5-cyclohexyl-2-imino-5-(4-methoxyphenyl)-3-methylimidazolidin-4-one;
5-(4-chlorophenyl)-5-cyclohexyl-2-imino-3-methylimidazolidin-4-one;
2-amino-5-cyclohexyl-5-(3-cyclopentyl phenyl)-3-methyl-3,5-dihydro-4H-imidazol-4-one;
2-amino-5-cyclohexyl-5-(3-cyclohexylphenyl)-3-methyl-3,5-dihydro-4H-imidazol-4-one;
5-[3-(benzyloxy)phenyl]-5-cyclohexyl-2-imino-3-methylimidazolidin-4-one;
N-{3-[2-amino-4-(4-methoxy-3-methylphenyl)-1-methyl-5-oxo-4,5-dihydro-1H-imidazol -4-yl]phenyl}-2-(4-chlorophenoxy)-2-methylpropanamide;
N-[3-(2-amino-4-cyclohexyl-1-methyl-5-oxo-4,5-dihydro-1H-imidazol-4-yl)phenyl]-3-methoxybenzamide;
N-[3-(2-amino-4-cyclohexyl-1-methyl-5-oxo-4,5-dihydro-1H-imidazol-4-yl)phenyl]-3-methoxypropanamide;
(2R)-N-[3-(2-amino-4-cyclohexyl-1-methyl-5-oxo-4,5-dihydro-1H-imidazol-4-yl)phenyl]-2-methoxy-2-phenylacetamide;
N-[3-(2-amino-4-cyclohexyl-1-methyl-5-oxo-4,5-dihydro-1H-imidazol-4-yl)phenyl]-3-methyl-2-furamide;
N-[3-(2-amino-4-cyclohexyl-1-methyl-5-oxo-4,5-dihydro-1H-imidazol-4-yl)phenyl]-2-(2-methoxyethoxy)acetamide;
N˜1˜-[3-(2-amino-4-cyclohexyl-1-methyl-5-oxo-4,5-dihydro-1H-imidazol-4-yl)phenyl]-N˜2˜,N˜2˜-dimethylglycinamide;
N-[3-(2-amino-4-cyclohexyl-1-methyl-5-oxo-4,5-dihydro-1H-imidazol-4-yl)phenyl]-3-(dimethylamino)benzamide;
N-[3-(2-amino-4-cyclohexyl-1-methyl-5-oxo-4,5-dihydro-1H-imidazol-4-yl)phenyl]-4-(dimethylamino)butanamide;
N-[3-(2-amino-4-cyclohexyl-1-methyl-5-oxo-4,5-dihydro-1H-imidazol-4-yl)phenyl]-1-methylpiperidine-4-carboxamide;
N-[3-(2-amino-4-cyclohexyl-1-methyl-5-oxo-4,5-dihydro-1H-imidazol-4-yl)phenyl]-2-cyclopropylacetamide;
N-[3-(2-amino-4-cyclohexyl-1-methyl-5-oxo-4,5-dihydro-1H-imidazol-4-yl)phenyl]-2-phenoxypropanamide;
N-[3-(2-amino-4-cyclohexyl-1-methyl-5-oxo-4,5-dihydro-1H-imidazol-4-yl)phenyl]-3-(trifluoromethyl)benzamide;
N-[3-(2-amino-4-cyclohexyl-1-methyl-5-oxo-4,5-dihydro-1H-imidazol-4-yl)phenyl]-2-(2-methoxyphenyl)acetamide;
N-[3-(2-amino-4-cyclohexyl-1-methyl-5-oxo-4,5-dihydro-1H-imidazol-4-yl)phenyl]-1-methyl-1H-pyrrole-2-carboxamide;
N-[3-(2-amino-4-cyclohexyl-1-methyl-5-oxo-4,5-dihydro-1H-imidazol-4-yl)phenyl]-2-methoxyacetamide;
N-[3-(2-amino-4-cyclohexyl-1-methyl-5-oxo-4,5-dihydro-1H-imidazol-4-yl)phenyl]-2-furamide;
N-[3-(2-amino-4-cyclohexyl-1-methyl-5-oxo-4,5-dihydro-1H-imidazol-4-yl)phenyl]-2-(benzyloxy)acetamide;
N-[3-(2-amino-4-cyclohexyl-1-methyl-5-oxo-4,5-dihydro-1H-imidazol-4-yl)phenyl]-2-methoxybenzamide;
N-[3-(2-amino-4-cyclohexyl-1-methyl-5-oxo-4,5-dihydro-1H-imidazol-4-yl)phenyl]-3,4-dimethoxybenzamide;
N-[3-(2-amino-4-cyclohexyl-1-methyl-5-oxo-4,5-dihydro-1H-imidazol-4-yl)phenyl]-2-(2,5-dimethoxyphenyl)acetamide;
(2E)-N-[3-(2-amino-4-cyclohexyl-1-methyl-5-oxo-4,5-dihydro-1H-imidazol-4-yl)phenyl]but-2-enamide;
N-[3-(2-amino-4-cyclohexyl-1-methyl-5-oxo-4,5-dihydro-1H-imidazol-4-yl)phenyl]butanamide;
N-[3-(2-amino-4-cyclohexyl-1-methyl-5-oxo-4,5-dihydro-1H-imidazol-4-yl)phenyl]-2-(3-methoxyphenyl)acetamide;
N-[3-(2-amino-4-cyclohexyl-1-methyl-5-oxo-4,5-dihydro-1H-imidazol-4-yl)phenyl]-1,3-benzodioxole-5-carboxamide;
N-[3-(2-amino-4-cyclohexyl-1-methyl-5-oxo-4,5-dihydro-1H-imidazol-4-yl)phenyl]-2-(4-chlorophenoxy)-2-methylpropanamide;
or a tautomer thereof or a stereoisomer thereof or a pharmaceutically acceptable salt thereof.

Compounds of the invention may be prepared employing conventional methods that utilize readily available reagents and starting materials. The reagents used in the preparation of the compounds of this invention can be either commercially obtained or can be prepared by standard procedures described in the literature. Representative compounds of the present invention can be prepared using the following synthetic schemes. The skilled practitioner will know how to make use of variants of these reaction sequences, which in themselves are well known in the art. For example, compounds of formula I wherein W is CO and R₁ and R₂ are H (Ia) may be prepared according to the following synthetic scheme illustrated in Flow Diagram I.

### Flow Diagram I

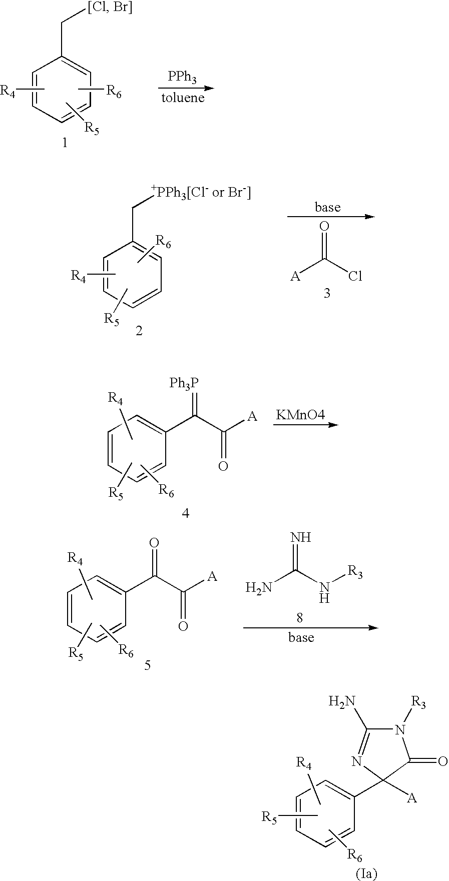

In flow diagram I, benzyl bromide or benzyl chloride 1 is converted to phosphonium salt 2 upon treatment with triphenylphospine and a non-polar solvent, i.e. toluene. The phosphonium salt 2 is first treated with a base, i.e. alkyllithium, sodium hydride, potassium tert-butoxide, in a solvent that does not adversely affect the subsequent addition of acid chloride 3, i.e. toluene, ether, tetrahydrofuran, and subsequently the generated anion is treated with carbocyclic acid chloride 3 to produce the corresponding ylide 4. The acid chloride 3 is either obtained commercially or prepared from the corresponding carboxylic acid upon treatment with a chlorinating agent such as oxalyl chloride or thionyl chloride. The ylide 4 is oxidized with potassium permanganate in the presence of magnesium sulfate in a polar or non-polar solvent, i.e. toluene, tetrahydrofuran, acetone to furnish diketone 5. Condensation of the substituted guanidine 8 with diketone 5 in the presence of an inorganic base, i.e. sodium carbonate, in a polar solvent, i.e ethyl alcohol, dioxane, N,N-dimethylformamide affords the desired compound of formula Ia.

Alternatively, compounds of formula Ia may be prepared as shown in Flow Diagram II wherein Et represents ethyl and Me represents methyl.

### Flow Diagram II

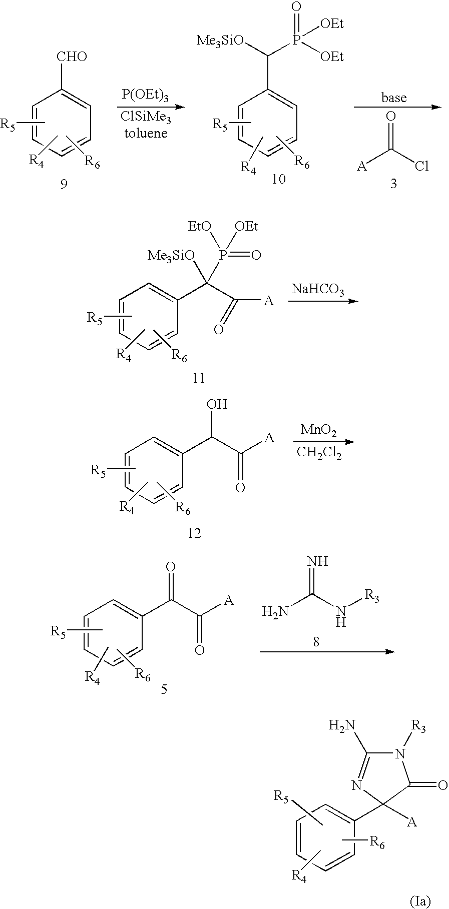

In flow diagram II, benzaldehyde 9 is treated with triethylphosphite and chlorotrimethylsilane in an aprotic solvent, i.e., toluene, to produce silyl-ether 10. Compound 10 is treated sequentially with a base, i.e., lithium diisopropylamide, and an acid chloride 3 to give sililoxy ether 11. Hydrolysis of said sililoxy ether in an aqueous inorganic base, i.e., sodium bicarbonate, affords alcohol 12. Compound 12 is oxidized with manganese dioxide to produce diketone 5. Condensation of said diketone with the substituted guanidine 8 in the presence of an inorganic base, i.e. sodium carbonate, in a polar solvent, i.e, ethyl alcohol, dioxane or N,N-dimethylformamide affords the desired compound of formula Ia.

Alternatively, the compounds of formula Ia may be prepared according to the synthetic scheme shown in flow diagram III.

### Flow Diagram III

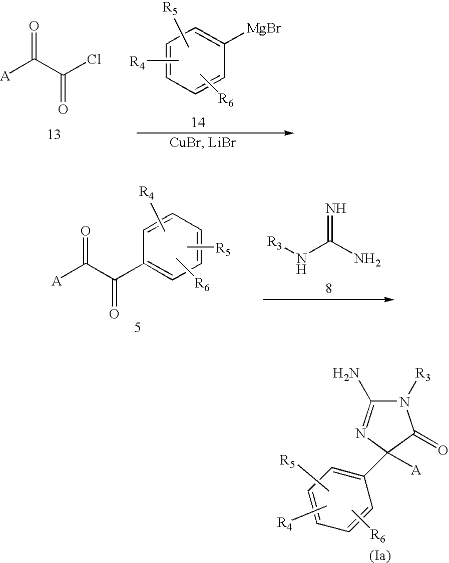

In flow diagram II, the Grignard reagent 14 is generated in situ and reacted with the glycolic acid chloride 13 to give the diketone 5. Said diketone is condensed with the substituted guanidine 8, as described hereinabove in flow diagrams I and II, to provide the desired compound of formula Ia.

Compounds of formula I wherein W is CO; R₁ and R₂ are H and R₅ is aryl or heteroaryl (Ib) may be prepared according to the following synthetic scheme illustrated in Flow Diagram IV wherein Hal represents Cl, Br or I.

### Flow Diagram IV

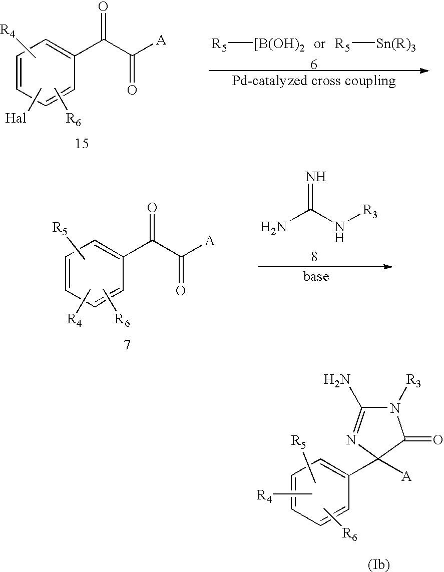

In flow diagram IV, the diketone 15 is employed in a palladium-catalyzed cross coupling reaction (Suzuki, Stille) with a heteroaryl or aryl boronic acid or a heteroaryl or aryl trialykl/triaryl stannate 6 in the presence of a variety of Pd(0) or Pd(II) catalysts, such as dichlorobis(tri-o-tolylphosphine)palladium(II), Pd(OAc)₂/tri-o-tolylphosphine, tetrakis(triphenylphosphine)palladium(0), or the like in a non-polar or polar solvent, i.e. toluene, diethoxyethyl ether, dioxane, or for the Suzuki reactions in the presence of inorganic bases, i.e. potassium carbonate to afford the diketone 7 wherein R₅ is a heteroaryl or aryl group. Condensation of the substituted guanidine 8 with diketone 7 as described in flow diagram I affords the desired compound of formula Ib.

The substituted guanidine 8 may be prepared using conventional methods, such as the reaction of 1-H-pyrazole-1-carboxamidine hydrochloride with a primary amine, R₃NH₂.

Compounds of formula I wherein A is adamantyl; R₁ and R₂ are H; and R₅ is a substituted phenyl group (Ic) may be prepared as shown in flow diagram V wherein R represents one or more optional substituents.

### Flow Diagram V

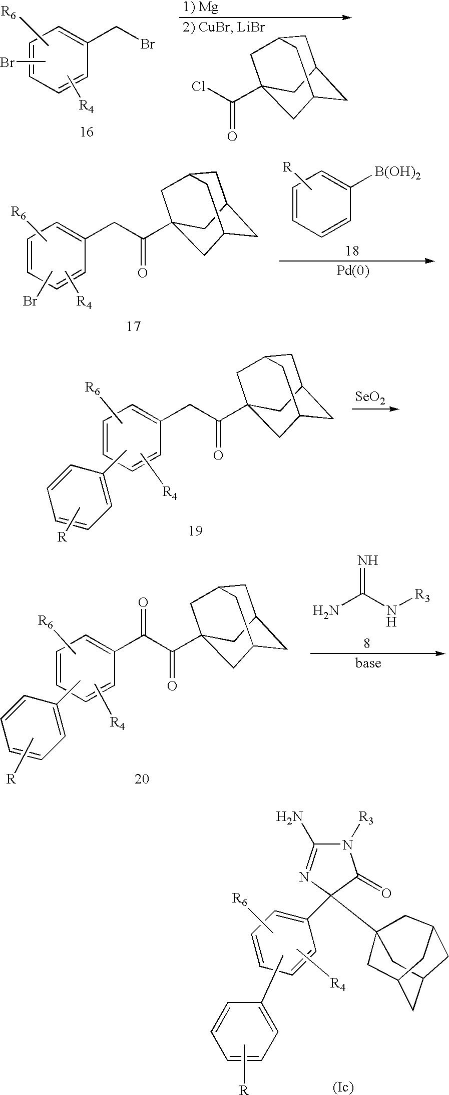

In flow diagram V, bromobenzyl bromide 16 is treated with magnesium turnings to give the corresponding Gringnard reagent, which is treated in situ with 1-adamantoyl chloride to give ketone 17. Suzuki coupling of 17 with boronic acid 18 yields the biphenyl compound 19. The biphenyl 19 is oxidized with selenium dioxide to give the corresponding diketone 20. Condensation of the diketone 20 with a substituted guanidine 8 as described hereinabove in flow diagram I affords the desired compound of formula Ic.

Compounds of formula I wherein W is CO; R₁, R₂, R₄ and R₆ are H and R₅ is cyclohexyl or cyclopentyl (Id) may be prepared as shown in flow diagram VI wherein m is 1 or 2.

### Flow Diagram VI

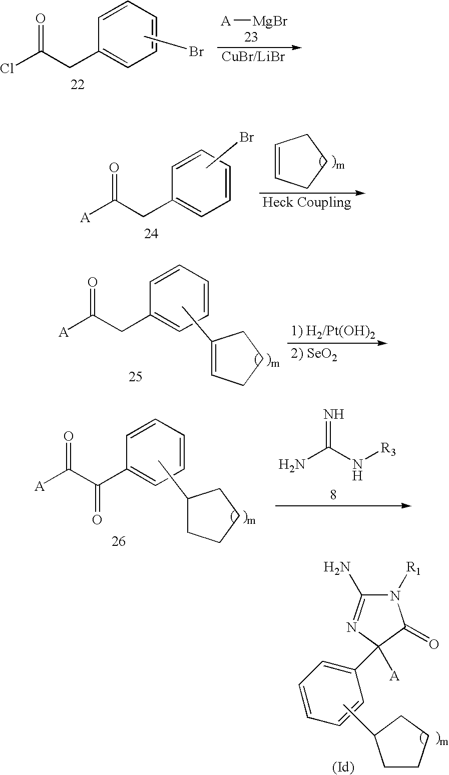

In flow diagram VI, the bromophenylacetyl chloride 22 is reacted with an in situ generated Grignard reagent 23 to give the ketone 24. Heck coupling of said ketone with cyclopentene or cyclohexene provides compound 25. Hydrogenation of compound 25, followed by oxidation with selenium dioxide gives the diketone 26. Condensation of compound 26 with the substituted guanidine 8 yields the desired compound of formula Id.

Compounds of formula I wherein W is CO and R₁ and R₂ are H (Ia) may also be prepared according to the synthetic scheme shown in flow diagram VII.

### Flow Diagram VII

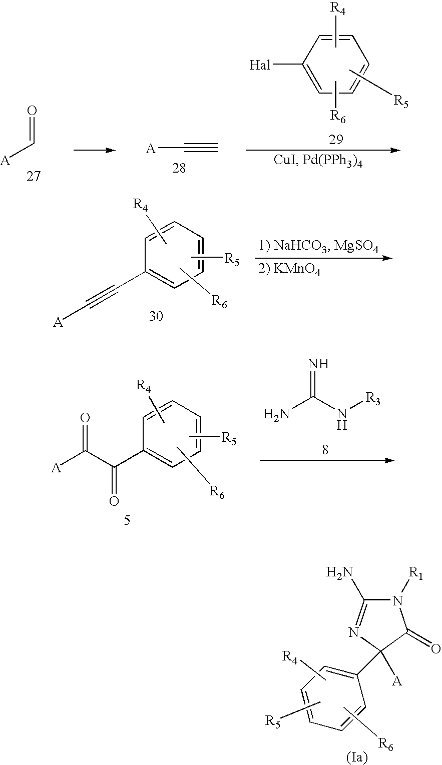

In flow diagram VII, a cycloalkyl aldehyde 27 is reacted with dimethyl(1-diazo-2-oxopropyl)phosphonate in the presence of K₂CO₃ and methanol to give the alkyne 28. Compound 28 is coupled with a substituted halobenzene 29 to give compound 30. Compound 30 is reacted with aqueous NaHCO₃ and MgSO₄, followed by treatment with KMnO4 to give the diketone 5. The diketone is then condensed with the guanidine 8, as described hereinabove, to give the desired compound of formula Ia.

Compounds of formula I wherein W is CS (Ie) may be readily prepared using conventional procedures, such as reacting a compound of formula Ia with CS₂ in the presence of a solvent to obtain the desired compound of formula Ie. Similarly, compounds of formula I wherein W is CH₂ (If) may be prepared by reacting a compound of formula Ia with a suitable reducing agent such as SnCl₂ to obtain the desired compound of formula If. The reactions are shown in flow diagram VIII.

### Flow Diagram VIII

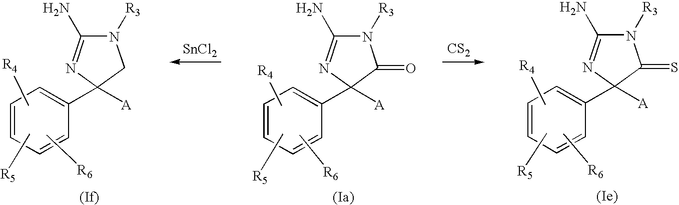

Compounds of formula I wherein R₁ and R₂ are other than H may be readily prepared by using conventional procedures such as reacting a compound of formula Ia, Ie or If with an alkylating agent, such as an alkyl halide, to give the compound of formula I wherein R₁ and R₂ are other than H. By using either one equivalent, or two or more equivalents, of the alkylating agent, compounds of formula I wherein R₁ is other than H and R₂ is H or wherein R₁ and R₂ are other than H may be obtained.

Advantageously, the compounds of formula I act as BACE inhibitors for the treatment or prevention of β-amyloid deposits and neurofibrillary tangles associated with such diseases as Alzheimer's disease, Trisomy 21 (Down's Syndrome), Hereditary Cerebral Hemorrhage with Amyloidosis of the Dutch-type (HCHWA-D), and other neurodegenerative disorders. Accordingly, the present invention provides methods for modulating BACE and treating, preventing, or ameliorating β-amyloid deposits and neurofibrillary tangles associated with diseases and disorders such as Alzheimer's disease, Trisomy 21 (Down's Syndrome), Hereditary Cerebral Hemorrhage with Amyloidosis of the Dutch-type (HCHWA-D), and other neurodegenerative disorders. Such methods generally involve administering to a patient suspected of suffering from or being susceptible to the disease or injury an effective amount of a compound of formula I. Also according to the present invention there is provided a method of treating Alzheimer's disease and related senile dementia's in humans or other mammals which comprises administering to a human or other mammal an effective amount of a compound of the present invention.

The present invention also provides methods for modulating (and, preferably, inhibiting) the activity of BACE, comprising administering to a patient and/or contacting a receptor thereof with an effective amount of at least one compound of Formula I. Certain methods further comprise determining BACE activity, either before or after said contacting step.

The present invention also provides methods of ameliorating β-amyloid deposits in a mammal, comprising administering to said mammal an effective amount of at least one compound of Formula I. Further methods ameliorate neurofibrillary tangles in a mammal, and comprise administering to said mammal an effective amount of at least one compound of Formula I.

Also provided are methods of ameliorating symptoms of Alzheimer's disease, cognitive impairment, Down's Syndrome, HCHWA-D, cognitive decline, senile dementia, cerebral amyloid angiopathy, degenerative dementia, or other neurodegenerative disorders in a mammal, comprising administering to said mammal an effective amount of at least one compound of Formula I.

As used in accordance with this invention, the term “providing,” with respect to providing a compound or substance covered by this invention, means either directly administering such a compound or substance, or administering a prodrug, derivative, or analog which will form the effective amount of the compound or substance within the body. This invention also covers providing the compounds of this invention to treat the disease states disclosed herein that the compounds are useful for treating.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The term “patient”, as used herein, refers to a mammal, preferably a human.

The terms “effective amount”, “therapeutically effective amount” and “effective dosage” as used herein, refer to the amount of a compound that, when administered to a patient, is effective to at least partially ameliorate (and, in preferred embodiments, cure) a condition from which the patient is suspected to suffer. It is understood that the effective dosage of the active compounds of this invention may vary depending upon the particular compound utilized, the mode of administration, the condition, and severity thereof, of the condition being treated, as well as the various physical factors related to the individual being treated. For treating Alzheimer's disease and other related senile dementia's, generally, satisfactory results may be obtained when the compounds of this invention are administered to the individual in need at a daily dosage of from about 0.1 mg to about 1 mg per kilogram of body weight, preferably administered in divided doses two to six times per day, or in a sustained release form. For most large mammals, the total daily dosage is from about 3.5 mg to about 140 mg preferably from about 3.5 to about 5 mg. In the case of a 70 kg human adult, the total daily dose will generally be from about 7 mg to about 70 mg and may be adjusted to provide the optimal therapeutic result. This regimen may be adjusted to provide the optimal therapeutic response.

The present invention also provides a pharmaceutical composition which comprises an effective amount of a compound of formula I and a pharmaceutically acceptable carrier.

The term “carrier”, as used herein, shall encompass carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, thransdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

In certain embodiments, the present invention is directed to prodrugs. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved.

For a more clear understanding, and in order to illustrate the invention more clearly, specific examples thereof are set forth hereinbelow. The following examples are merely illustrative and are not to be understood as limiting the scope and underlying principles of the invention in any way. Various modifications of the invention, in addition to those described herein, will be apparent to those skilled in the art from the foregoing description. Such modifications are also intended to fall within the scope of the appended claims.

Unless otherwise stated, all parts are parts by weight. The following abbreviations are used: Ph represents phenyl, TEA is triethylamine, DMSO is dimethylsulfoxide, DMF is N,N-dimethylformamide, NMR is proton nuclear magnetic resonance, and MS is mass spectroscopy with (+) referring to the positive mode which generally gives a M+1 (or M+H) absorption where M=the molecular mass. All compounds are analyzed at least by MS and NMR.

### EXAMPLE 1

### Preparation of 2-Amino-5-bicyclo[2.2.1]hept-1-yl-5-(4-methoxy-3-methylphenyl) -3-methyl-3,5-dihydro-4H-imidazol-4-one

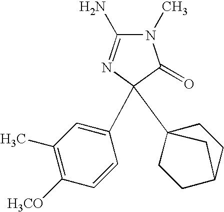

### Step a) Preparation of bicyclo[2.2.1]heptane-1-carboxylic acid

The compound was synthesized according to the procedure described in Reike, R. D.; Bales, S. E.; Hundnall, P. M.; Burns, T. P.; Poindexter, G. S.; Org Syn, 1988 (VI) 845.

### Step b) Preparation of Bicyclo[2.2.1]heptane-1-carbonyl chloride

A solution of bicyclo[2.2.1]heptane-1-carboxylic acid (0.3 g, 2.1 mmol) in thionyl chloride plus 1 drop of DMF was heated at reflux temperature for 3 h and concentrated to dryness under reduced pressure. The residue was used without any further purification.

### Step c) Preparation of 3-Methyl,4-methoxy-benzyl triphenylphosphine chloride

A solution of triphenylphosphine (3 g, 11.4 mmol) in toluene was treated with 3-methyl-4-methoxybenzylchloride (1.9 g, 11.4 mmol), heated at reflux temperature for 3 h, cooled to room temperature and filtered. The filtercake was washed with a small amount of ether, and dried to afford a white solid, 1.9 g (33% yield), MS m/e (M)⁺ 397; ¹H NMR (DMSOd₆ 300 MHz).δ1.9 (s, 3H), 3.72 (s, 3H), 5.3 (d, 2H), 6.55 (s, 1H), 6.82 (m, 2H), 7.65 (t, 6H), 7.75 (m, 6H), 7.92 (t, 3H).

### Step d) Preparation of 1-Bicyclo[2.2.1]hept-1-yl-2-(4-methoxy-3-methyl-phenyl)-ethane-1,2-dione

A solution of 3-methyl-4-methoxybenzyl triphenylphosphonium chloride (1.8 g, 4.2 mmol) in toluene was treated with n-BuLi 2.5 N (4.2 mmol), stirred for 15 min at room temperature, treated in one portion with a toluene solution of bicyclo[2.2.1]heptane-1-carbonyl chloride (0.33 g, 2.1 mmol), stirred at room temperature for 3 hr and filtered. The filtrate was diluted with water, treated with KMnO₄ (0.5 g, 4.2 mmol) and MgSO₄ (1 g, 8.4 mmol), heated at 60° C. for 16 h, cooled to room temperature and filtered. The filtercake was washed with ether and water. The washes were combined with the filtrate and the phases were separated. The organic phase was dried over MgSO₄ and concentrated in vacuo. The resultant residue was purified by flash chromatography on silica gel in ethyl acetate/hexane 20:1 to afford the title diketone as a yellow oil, 0.04 g, MS m/e (M)⁺ 272 ¹H NMR (DMSOd₆ 300 MHz).δ1.3 (m, 2H), 1.6 (m, 6H), 2.1 (s, 3H), 2.3 (m, 1H), 3.8 (s, 3H), 7.1 (d, 1H), 7.6 (m, 2H).

### Step e) Preparation of 2-amino-5-bicyclo[2.2.1]hept-1-yl-5-(4-methoxy-3-methylphenyl)-3-methyl-3,5-dihydro-4H-imidazol-4-one

A solution of 1-bicyclo[2.2.1]hept-1-yl-2-(4-methoxy-3-methylphenyl)ethane-1,2-dione (0.40 g, 0.14 mmol) in ethanol (3 mL) and water (1 mL) was treated with 1-methylguanidine hydrochloride (0.05 g, 0.44 mmol) followed by K₂CO₃ (0.06 g, 0.44 mmol), heated at reflux temperature for 3 h, cooled to room temperature and concentrated to remove the ethanol. The remaining aqueous mixture was diluted with water and extracted with CHCl₃. The organic extracts were combined, dried over MgSO₄ and concentrated in vacuo. The resultant residue was purified by flash chromatography on silica gel in 10% methanol/ethyl acetate to give the title product as a white solid, 0.01 g (24% yield); ¹HNMR (DMSO-d₆, 300 MHz) δ 1.2 (m, 7H), 1.6 (m, 1H), 2.05 (m, 1H), 2.1 (s, 3H), 2.95 (s, 3H), 3.8 (s, 3H), 6.4 (b, 2H), 6.8 (d, 2H), 7.4 (m, 3H); MS m/e 328 (M)⁺

### EXAMPLE 2

### Preparation of 5-(1-Adamantyl)-2-amino-5-(4-methoxyphenyl)-3-methyl-3,5-dihydro-4H-imidazol-4-one

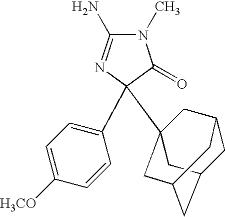

### Step a) Preparation of 1-(1-Adamantyl)-2-(4-methoxyphenyl)ethane-1,2-dione

A solution of 4-methoxybenzyl triphenylphosphonium chloride (4.19 g, 10 mmol) in toluene was treated with n-BuLi 2.5 N (10 mmol), stirred for 15 min at room temperature, treated with a toluene solution of 1-adamantanecarbonyl chloride (1 g, 5 mmol) in one portion, stirred at room temperature for 3 hr and concentrated to dryness in vacuo. The residue was dissolved in a mixture of water and toluene, treated with KMnO₄ (1.58 g, 10 mmol) and MgSO₄ (4.8 g, 40 mmol), heated at 60° C. for 16 h, cooled to room temperature and filtered. The filtercake was washed with ether and water. The washes were combined with the filtrate and the phases were separated. The organic phase was dried over MgSO₄ and concentrated in vacuo. The resultant residue was purified by flash chromatography on silica gel in ethyl acetate/hexane 20:1 to afford the title diketone as a yellow oil, 0.15 g, MS m/e 298 (M)⁺; ¹HNMR (DMSO-d₆, 300 MHz) δ 1.7 (m, 6H), 1.8 (m, 6H), 2.0 (m, 3H), 2.4 (s, 1H), 3.8 (s, 3H), 7.1 (d, 2H), 7.7 (d, 2H).

### Step b) Preparation of 5-(1-Adamantyl)-2-amino-5-(4-methoxyphenyl)-3-methyl-3,5-dihydro-4H-imidazol-4-one

Using essentially the same procedure described in Example 1, step e, and employing 1-(1-adamantyl)-2-(4-methoxyphenyl)ethane-1,2-dione and 1-methylguanidine hydrochloride, the title product is obtained as a white solid, 0.04 g (26% yield), MS m/e 352 (M)⁺; ¹HNMR (DMSO-d₆, 300 MHz) δ 1.4 (m, 6H), 1.5 (m, 3H), 1.6 (m, 3H), 1.9 (m, 3H), 2.95 (s, 3H), 3.7 (s, 3H), 6.4 (b, 2H), 6.8 (d, 2H), 7.5 (d, 2H).

### EXAMPLE 3

### Preparation of 5-(1-Adamantyl)-2-amino-5-(4-ethoxyphenyl)-3-methyl-3,5-dihydro-4H-imidazol-4-one

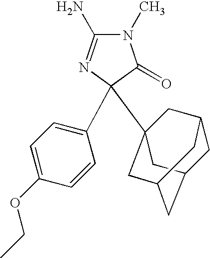

Using essentially the same procedures described in Example 2, step a and Example 1, step e, and employing 1-(1-adamantyl)-2-(4-ethoxyphenyl)ethane-1,2-dione and 1-methylguanidine hydrochloride, the title product was obtained as a white solid, MS m/e 368 (M)⁺; ¹HNMR (DMSO-d₆, 300 MHz) 61.2 (t, 3H), 1.3 (m, 6H), 1.5 (m, 3H), 1.6 (m, 3H), 1.8 (m, 3H), 2.8 (s, 3H), 4.0 (t, 2H), 6.4 (b, 2H), 6.8 (d, 2H), 7.5 (d, 2H).

### EXAMPLE 4

### Preparation of 5-(1-Adamantyl)-2-amino-5-(4-butoxyphenyl)-3-methyl-3,5-dihydro-4H-imidazol-4-one

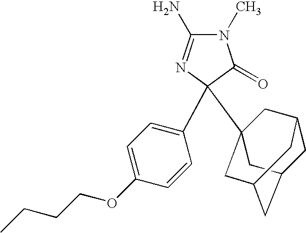

Using essentially the same procedures described in Example 2, step a and Example 1, step e, and employing 1-(1-adamantyl)-2-(4-butoxyphenyl)ethane-1,2-dione and 1-methylguanidine hydrochloride, the title product was obtained as a white solid, MS m/e 396 (M)⁺; ¹HNMR (DMSO-d₆, 300 MHz) δ 0.9 (t, 3H), 1.4 (m, 8H), 1.6 (m, 3H), 1.7 (m, 5H), 1.9 (m, 3H), 2.9 (s, 3H), 3.9 (t, 2H), 6.4 (b, 2H), 6.8 (d, 2H), 7.5 (d, 2H).

### EXAMPLE 5

### Preparation of 5-(1-Adamantyl)-2-amino-5-(3-ethyl-4-methoxyphenyl)-3-methyl -3,5-dihydro-4H-imidazol-4-one

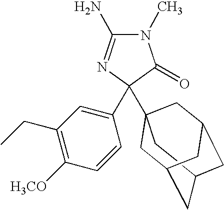 Using essentially the same procedures described in Example 2, step a and Example 1, step e, and employing 1-(1-adamantyl)-2-(3-ethyl-4-methoxyphenyl)ethane-1,2-dione and 1-methylguanidine hydrochloride, the title product was obtained as a white solid, MS m/e 382 (M)⁺; ¹HNMR (DMSO-d₆, 300 MHz) δ 1.0 (t, 3H), 1.4 (m, 7H), 1.5 (m, 3H), 1.6 (m, 3H), 1.8 (m, 3H), 2.4 (q, 2H), 2.8 (t, 2H), 3.7 (s, 3H), 6.4 (b, 2H), 6.8 (d, 1H), 7.4 (m, 2H).

### EXAMPLE 6

### Preparation of 5-(1-Adamantyl)-2-amino-5-(4-methoxy-3,5-dimethylphenyl)-3-methyl-3,5-dihydro-4H-imidazol-4-one

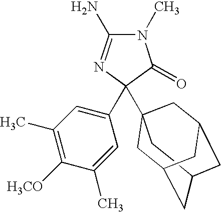

### Step a) Preparation of 5-Chloromethyl-2-methoxy-1,3-dimethylbenzene

A solution of 2-methoxy-1,3-dimethylbenzene (10 g, 73 mmol) in glacial acetic acid was treated with paraformaldehyde (12 g, 142 mmol), heated to 60° C. with HCl (gas) bubbled in for 4 h and cooled to room temperature. The reaction was poured into water and extracted with ether. The extracts were combined, washed sequentially with water, aqueous saturated sodium bicarbonate and brine, dried over MgSO₄ and concentrated in vacuo. The resultant residue was distilled (100° C. and 0.2 mmHg) to give the chloromethylbenzene compound as a clear oil, 2.3 g (17% yield), ¹HNMR (DMSO-d₆, 300 MHz) δ 2.2 (s, 6H), 3.6 (s, 3H), 4.6 (s, 2H), 7.0 (s, 2H).

### Step b) Preparation of (4-Methoxy-3,5-dimethyl-benzyl)-triphenyl-phosphonium chloride

Using essentially the same procedure described Example 1, step c, and employing 5-chloromethyl-2-methoxy-1,3-dimethyl-benzene and triphenylphosphine, the title phosphonium chloride was obtained as a white solid, MS m/e 411 (M)⁺; ¹HNMR (DMSO-d₆, 300 MHz) δ 1.9 (s, 6H), 3.3 (s, 3H), 5.0 (d, 2H), 7.6 (m, 6H), 7.7 (m, 6H), 7.9 (m, 3H).

### Step c) Preparation of 1-Adamantan-1-yl-2-(4-methoxy-3,5-dimethyl-phenyl)-ethane -1,2-dione

Using essentially the same procedure described in Example 2, step a, and employing (4-methoxy-3,5-dimethyl-benzyl)-triphenyl-phosphonium chloride and 1-adamantanecarbonyl chloride, the title diketone was obtained as a yellow oil, MS m/e 327 (M)⁺; ¹HNMR (DMSO-d₆, 300 MHz) 61.6 (m, 6H), 1.8 (m, 6H), 2.0 (m, 3H), 2.3 (s, 6H), 3.7 (s, 3H), 7.4 (s, 2H).

### Step d) Preparation of 5-(1-Adamantyl)-2-amino-5-(4-methoxy-3,5-dimethyl-phenyl) -3-methyl-3,5-dihydro-4H-imidazol-4-one

Using essentially the same procedure described in Example 1, step e, and employing 1-adamantan-1-yl-2-(4-methoxy-3,5-dimethylphenyl)ethane-1,2-dione and 1-methylguanididne, the title product was obtained as a white solid, MS m/e 382 (M)⁺; ¹HNMR (DMSO-d₆, 300 MHz) δ 1.4 (m, 6H), 1.5 (m, 3H), 1.7 (m, 3H), 1.8 (m, 3H), 2.1 (s, 6H), 2.8 (s, 3H), 3.6 (s, 3H), 6.4 (b, 2H), 7.2 (s, 2H).

### EXAMPLE 7

### Preparation of 5-(1-Adamantyl)-2-amino-3-methyl-5-phenyl-3,5-dihydro-4H-imidazol-4-one

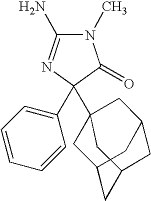

### Step a) Preparation of Diethyl 1-phenyl-1-(trimethylsilyloxy)methane phosphonate

Under a nitrogen atmosphere, into a cold (0° C.) solution of benzaldehyde (10.15 mL, 0.1 mmol) and triethylphosphite (19.1 mL, 0.1 mol) was added dropwise chlorotrimethylsilane (12.6 mL, 0.1 mol) over 10 minutes. After the addition was complete, the ice bath was removed and the reaction mixture was heated up to 120° C. (oil bath) for 8 hours and distilled (180° C., 10.0 mmHg) to afford the title compound as a colorless oil (25 g, 79% of yield); MS m/e (M+H)⁺ 317; ¹H NMR (400 MHZ, CDCl₃) δ 0.08 (s, 9H), 1.22 (m, 6H), 4.01 (m, 4H), 4.97 (d, 1H), 7.32 (m, 3H), 7.44 (m, 2H).

### Step b) Preparation of (2-Adamantan-1-yl-2-oxo-1-phenyl-1-trimethylsilanyloxy-ethyl)-phosphonic acid diethyl ester

Under a nitrogen atmosphere, into a cold (−78° C.) solution of diethyl 1-phenyl)[(trimethylsilyl)oxy]methyl}phosphonate (3.16 g, 10 mmol) in THF was added dropwise lithium diisopropylamide (2M, 5.25 mL) over 10 minutes. The reaction mixture was stirred for another 30 minutes, treated with adamantane-1-carboxylic acid chloride (2.09 g, 10 mmol) in THF, heated slowly to room temperature overnight. Under cooling the reaction mixture was poured into saturated NH₄Cl solution and extracted with ether. The extracts were combined, dried over MgSO4 and concentrated in vacuo. The resultant residue was purified by flash chromatography on silica gel (hexanes/EtOAc 95/5)) to afford the title diester as a colorless oil, 2.1 g (43% yield), MS m/e (M+H)⁺ 479.1; ¹H NMR (400 MHZ, DMSOd₆) δ 0.21 (s, 9H), 1.13 (t, 6H), 1.45-1.82 (m, 15H), 3.91 (dq, 4H), 7.30-7.43 (m, 5H).

### Step c) 1-Adamantan-1-yl-2-phenyl-ethane-1,2-dione

A mixture of (2-adamantan-1-yl-2-oxo-1-phenyl-1-trimethylsilanyloxyethyl)-phosphonic acid diethyl ester (2.1 g, 4.38 mmol), and a mixture of aqueous saturated NaHCO₃ solution (1:1) in methanol was heated at reflux temperature for 2 hours, cooled, acidified with 2N HCl and extracted with ether. The ether extracts were combined, dried over MgSO₄ and concentrated in vacuo. Purification of this residue by flash chromatography on silica gel (hexane/EtOAc 95/5) gave the title diketone as a yellow oil (0.21 g, 18% yield); MS m/e (M+)⁺ 268.15; ¹H NMR (400 MHZ, DMSO-d₆) δ 1.70 (m, 6H), 1.90 (bs, 6H), 2.02 (m, 3H), 7.62 (m, 2H,), 7.79 (m, 3H).

### Step d) Preparation of 5-(1-Adamantyl)-2-amino-3-methyl-5-phenyl-3,5-dihydro -4H-imidazol-4-one

A mixture of 1-adamantan-1-yl-2-phenylethane-1,2-dione (0.21 g, 0.78 mmol), Na₂CO₃ (0.25 g, 2.34 mmol), N-methylguanidine hydrochloride (0.12 g, 1,01 mmol), and H₂O (0.70 mL) in dioxane and EtOH was stirred at 80° C. for 18 hours and concentrated in vacuo. The resultant residue was dissolved in CHCl₃, washed with water, dried over K₂CO₃ and evaporated to dryness. Purification of this residue by flash chromatography on silica gel (EtOAc/CH₂Cl₂/Et₃N 7.5/2/0.5) gave th title product as a white solid (0.11 g, 43% yield, mp 250° C.); MS m/e (M+H)⁺ 324; ¹H NMR (400 MHZ, DMSO-d₆) δ 1.40 (m, 6H), 1.50 (m, 3H), 1.70 (m, 3H), 1.85 (bs, 3H), 2.85 (s, 3H), 6.40 (bs, 2H), 722 (m, 3H), 7.62 (m, 2H).

### EXAMPLE 8

### Preparation of 5-(1-Adamantyl)-2-amino-5-(4-methoxy-3-methylphenyl)-3-methyl-3,5-dihydro-4H-imidazol-4-one

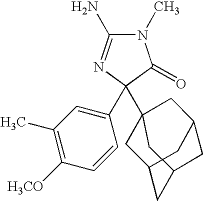

### Step a) Preparation of Diethyl {(4-methoxy-3 methylphenyl)[(trimethylsilyl)oxy]methyl}phosphonate

Under a nitrogen atmosphere, into a cold (0° C.) solution of 3-methyl-4-methoxy-benzaldehyde (15 g, 0.1 mol) and triethylphosphite (19.1 mL, 0.1 mol) was added dropwise chlorotrimethylsilane (12.6 mL, 0.1 mol) over 10 minutes, heated at 120° C. (oil bath) for 8 hours and distilled (180° C., 10.0 mmHg). The distillate was further purified by flash chromatography on silica gel (hexanes/ethyl acetate/isopropyl alcohol, 7/2.5/0.5) to give the title phophonate compound as a white solid (16 g, 44% of yield, mp 37° C.); MS m/e (M+H)⁺ 361; ¹H NMR (400 MHZ, DMSOd₆) δ 0.1 (s, 9H), 1.18 (dt, 6H), 2.1 (s, 3H), 3.68 (s, 3H), 3.85 (m, 4H), 4.97 (d, 1H), 6.87 (m, 1H), 7.20 (m, 2H).

### Step b) Preparation of 2-Adamantan-1-yl-1-(4-methoxy-3-methyl-phenyl)-2-oxo -1-trimethylsilanyloxy-ethyl]-phosphonic acid diethyl ester

Using substantially the same procedure described in the Example 7, step b, and employing diethyl {(4-methoxy-3-methylphenyl)[(trimethylsilyl)oxy]methyl}phosphonate (3.6 g, 10 mmol) and adamantane-1-carboxylic acid chloride (2.09 g, 10 mmol), the title diester compound was obtained as a yellow oil (0.98 g, 22% yield); MS m/e (M+H)⁺ 523.2; ¹H NMR (400 MHZ, DMSO-d₆) δ 0.21 (s, 9H), 1.15 (t, 6H), 1.45-1.82 (m, 15H), 2.17 (s, 3H), 3.78 (s, 3H), 3.91 (dq, 4H), 6.90 (d, 1H), 7.20 (m, 2H).

### Step c) Preparation of 1-Adamantan-1-yl-2-(4-methoxy-3-methyl-phenyl)-ethane -1,2-dione

Using substantially the same procedure described in Example 7, step c, and employing 2-adamantan-1-yl-1-(4-methoxy-3-methyl-phenyl)-2-oxo-1-trimethylsilanyloxy-ethyl]-phosphonic acid diethyl ester (0.32 g, 0.61 mmol), the title diketone was obtained as a yellow oil (0.21 g, 53% yield); MS m/e (M+H)⁺ 313.14; ¹H NMR (400 MHZ, CDCl₃) δ 1.69 (m, 6H), 1.93 (bs, 6H), 2.01 (bs, 3H), 2.21 (s, 3H), 3.87 (s, 3H), 6.85 (d, 1H,), 7.60 (m, 2H).

### Step d) Preparation of 5-(1-Adamantyl)-2-amino-5-(4-methoxy-3-methylphenyl) -3-methyl-3,5-dihydro-4H-imidazol-4-one

Using substantially the same procedure described in Example 7, step d, and employing 1-adamantan-1-yl-2-(4-methoxy-3-methyl-phenyl)-ethane-1,2-dione (0.21 g, 0.67 mmol), the title product was obtained as a white solid (0.105 g, 42% yield, mp 255° C.); MS m/e (M+H)⁺ 368; ¹H NMR (400 MHZ, DMSO-d₆) δ 1.40 (m, 6H), 1.55 (m, 3H), 1.70 (m, 3H), 1.85 (bs,3H) 2.10 (s, 3H), 2.85 (s, 3H), 3.78 (s, 3H), 6.30 (bs, 2H), 6.80 (d, 1H), 7.42 (m, 2H).

### EXAMPLE 9

### Preparation of (5S)-(1-Adamantyl)-2-amino-5-(4-methoxy-3-methylphenyl)-3-methyl-3,5-dihydro-4H-imidazol-4-one

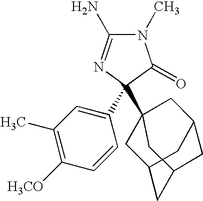

The racemate 5-(1-adamantyl)-2-amino-5-(4-methoxy-3-methylphenyl)-3-methyl-3,5-dihydro-4H-imidazol-4-one was separated by HPLC technique on Chiralcel AD, 046×25 cm using mobile phase EtOH:H₂O (6:4 with 0.1% DEA) in a flow rate of 1.0 mL/min. The title S-isomer was obtained after evaporation as a white solid; mp 376° C.; MS m/e (M+H)⁺ 368; ¹H NMR (400 MHZ, DMSO-d₆) δ 1.40 (m, 6H), 1.55 (m, 3H), 1.70 (m, 3H), 1.85 (bs,3H), 2.10 (s, 3H), 2.85 (s, 3H), 3.70(s, 3H), 6.40 (bs, 2H), 6.80 (d, 1H), 7.42 (m, 2H).

### EXAMPLE 10

### Preparation of (5R)-(1-Adamantyl)-2-amino-5-(4-methoxy-3-methylphenyl)-3-methyl-3,5-dihydro-4H-imidazol-4-one

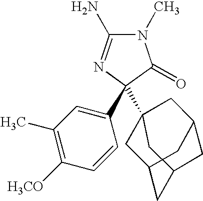

The racemate 5-(1-adamantyl)-2-amino-5-(4-methoxy-3-methylphenyl)-3-methyl-3,5-dihydro-4H-imidazol-4-one was separated by HPLC technique on Chiralcel AD, 046×25 cm using mobile phase EtOH:H₂O (6:4 with 0.1% DEA) in a flow rate of 1.0 mL/min. The title R-isomer was obtained, after evaporation, as a white solid; mp 376° C.; MS m/e (M+H)⁺ 368; ¹H NMR (400 MHZ, DMSO-d₆) δ 1.40 (m, 6H), 1.50 (m, 3H), 1.70 (m, 3H), 1.85 (bs,3H), 2.10 (s, 3H), 2.85 (s, 3H), 3.70 (s, 3H), 6.40 (bs, 2H), 6.80 (d, 1H), 7.42 (m, 2H).

### EXAMPLE 11

### Preparation of 5-(1-Adamantyl)-2-amino-5-(3,4-dimethoxyphenyl)-3-methyl-3,5-dihydro-4H-imidazol-4-one

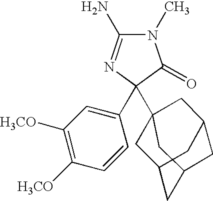

Using essentially the same procedures described in Example 7, steps a-d, and employing diethyl {(3,4-dimethoxyphenyl)[(trimethylsilyl)oxy]methyl)phosphonate as starting material, the title product was obtained as a white solid, 0.08 g (60% yield), mp 130° C.; MS m/e (M+H)⁺ 384; ¹H NMR (400 MHZ, DMSO-d₆) 61.40 (m, 6H), 1.50 (m, 3H), 1.70 (m, 3H), 1.85 (bs,3H), 2.85 (s, 3H), 3.65 (ds, 6H), 6.30 (bs, 2H), 6.80(d, 1H), 7.20 (m, 2H).

### EXAMPLE 12

### Preparation of 5-(1-Adamantyl)-2-amino-5-(4-methoxy-2.3 dimethylphenyl)-3-methyl-3,5-dihydro-4H-imidazol-4-one

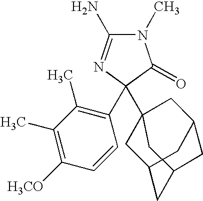

### Step a) Preparation of 1-Chloromethyl-4-methoxy-2,3-dimethyl-benzene

Into a cooled (0° C.) stirred solution of (4-methoxy-2,3-dimethyl-phenyl)-methanol (5 g, 30.08 mmol) in dioxane was added anhydrous ZnCl₂ (0.1 g), then SOCl₂ (4.4 ml, 60.16 mmol) in dioxane (6 ml) over 1 hour. The reaction mixture was allowed to come to room temperature and stirred for another hour. The volatiles were removed in vacuo and the residue was dissolved in ether and washed with saturated aqueous NaHCO₃ then dried over MgSO₄. Evaporation and purification by distillation gave the title chloromethylbenzene compound as a white solid (1.2 g, 22% yield). MS m/e (M)⁺ 184; ¹H NMR (400 MHZ, DMSO-d₆) δ 2.01 (s, 3H), 2.41 (s, 3H), 76 (s, 3H), 4.78 (s, 2H), 6.77 (d, 1H), 7.19 (d, 1H).

### Step b) Preparation of 4-Methoxy-2,3-dimethylbenzyltriphenylphosphonium chloride

A solution of 1-chloromethyl-4-methoxy-2,3-dimethyl-benzene (1.02 g, 5.52 mmol) and triphenylphosphine (1.46 g, 5.57 mmol) in toluene was heated at reflux temperature for 18 hours. The resulting white suspension was diluted with hexane and filtered. The filtercake was washed with hexane and to give the title phosphonium chloride as a white solid (1.93 g, 80% yield); MS m/e (M)⁺ 411; ¹H NMR (400 MHZ, DMSO-d₆) δ 1.38 (s, 3H), 1.88 (s, 3H), 3.67 (s, 3H), 4.99 (d, 2H), 6.66 (d, 1H), 6.77 (d, 1H), 7.58 (m, 6H), 7.68 (m, 6H), 7.86 (m, 3H).

### Step c) Preparation of 1-Adamantan-1-yl-2-(4-methoxy-2,3,dimethylphenyl)-ethane -1,2-dione

Under a nitrogen atmosphere, a cold (0° C.) suspension of 4-methoxy-2,3-dimethyl-benzyl-triphenyl-phosphonium chloride (1.90 g, 4.3 mmol) in toluene was treated dropwise with n-BuLi (2.5 M in hexane, 1.8 mL) over a 10 minute period, heated up to room temperature, stirred for 80 minutes, cooled to 0° C., treated with a solution of freshly prepared adamantane-1-carboxylic acid chloride (0.427 g, 2.15 mmol) in toluene, warmed to room temperature, stirred for 18 hours and concentrated in vacuo. The resultant residue was dissolved in a mixture of H₂O and acetone, treated with MgSO₄ (2.0 g) and KMnO₄ (1.3 g), stirred at 50° C. for 18 hours, with ether and H₂O and filtered through solka floc. The filtrate was separated; the organic phase was dried over MgSO₄ and evaporated to dryness. This residue was and purified by flash chromatography on silica gel (hexanes/EtOAc 95/5), to give the title diketone compound as an off white solid (0.21 g, 30% yield, mp 140° C.); MS m/e (M+H)⁺ 327.1; ¹H NMR (400 MHZ, CDCl₃) δ1.70 (m, 6H), 1.95 (m, 6H), 2.02 (m, 3H), 2.17 (s, 3H), 2.57 (s, 3H), 3.85 (s, 3H), 6.72 (d, 1H), 7.29 (d, 1H).

### Step d) Preparation of 5-(1-Adamantyl)-2-amino-5-(4-methoxy-2,3-dimethylphenyl)-3-methyl-3,5-dihydro-4H-imidazol-4-one

Using substantially the same procedure described in Example 7, step d, and employing 1-adamantan-1-yl-2-(4-methoxy-2,3,dimethylphenyl)ethane-1,2-dione (0.19 g, 0.582 mmol) and methylguanidine hydrochloride, the title product was obtained as a white solid (0.12 g, 54% yield, mp 295° C.); MS m/e (M−H)⁻ 380; ¹H NMR (400 MHZ, DMSO-d₆) δ 1.40 (m, 6H), 1.50 (m, 3H), 1.70 (m, 3H), 1.80 (bs,3H), 2.00 (s, 3H), 2.60 (s, 3H), 2.80 (s, 3H), 3.65 (s, 3H), 6.20 (bs, 2H,),6.60 (d, 1H), 7.85 (d, 1H).

### EXAMPLE 13

### Preparation of 2-Amino-5-bicyclo[2.2.1]hept-2-yl-5-(4-methoxy-3-methylphenyl) -3-methyl-3,5-dihydro-4H-imidazol-4-one

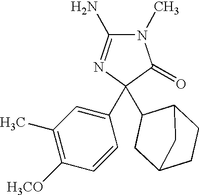

Using essentially the same procedure described in Example 7, step d, and employing 1-bicyclo[2.2.1]hept-2-yl-2-(4-methoxy-3-methylphenyl)ethane-1,2-dione and methylguanidine hydrochloride, the title product was obtained as a white solid (0.175 g, 34% yield, mp 121° C.); MS m/e (M+H)⁺ 326; ¹H NMR (400 MHZ, DMSO-d₆) δ 0.85-1.40 (m, 8H), 1.60 (m, 1H), 1.70 (m, 1H), 2.01 (bs, 1H), 2.10 (s, 3H), 2.90 (s,3H), 3.78 (s, 3H), 6.40 (bs, 2H), 6.81 (d, 1H), 7.30 (m, 2H).

### EXAMPLE 14

### Preparation of 2-Amino-5-hexahydro-2,5-methanopentalen-3a(1H)-yl-5-(4-methoxy-3-methylphenyl)-3-methyl-3,5-dihydro-4H-imidazol-4-one

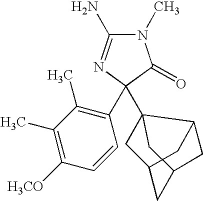

### Step a) Preparation of [2-(Hexahydro-2,5-methano-pentalen-3a-yl)-1-(4-methoxy-3-methyl-phenyl)-2-oxo-1-trimethylsilanyloxy-ethyl]-phosphonic acid diethyl ester

Using substantially the same procedure described in Example 7, step b, and employing diethyl {(4-methoxy-3-methylphenyl)[(trimethylsilyl)oxy]methyl}-phosphonate (3.61 g, 10 mmol) and bicyclo[3.3.1]nonane-3-carboxylic acid chloride (10 mmol), the title diester was obtained as a yellow oil (2.2 g, 42% yield); MS m/e (M+H)⁺ 509.2; ¹H NMR (400 MHZ, DMSO-d₆) δ 0.25 (s, 9H), 1.15 (t, 3H), 1.22 (t, 3H), 1.36-2.03 (m, 13H), 2.18 (s, 3H), 3.80 (s, 3H), 4.04 (m, 4H), 6.76 (d, 1H), 7.29 (m, 2H).

### Step b) Preparation of 1-(Hexahydro-2,5-methano-pentalen-3a-yl)-2-(4-methoxy -3-methyl-phenyl)-ethane-1,2-dione

Using substantially the same procedure described in Example 7, step c, and employing [2-(hexahydro-2,5-methano-pentalen-3a-yl)-1-(4-methoxy-3-methyl-phenyl) -2-oxo-1-trimethylsilanyloxy-ethyl]-phosphonic acid diethyl ester (1.1 g, 2.17 mmol) and replacing the methanol by dioxane, the title diketone compound was obtained as a yellow oil (0.125 g, 20% yield);); MS m/e (M+H)⁺ 299.4; ¹H NMR (400 MHZ, CDCl₃) δ 1.59-1.61 (m, 5H), 1.80 (m, 4H), 2.18 (m, 2H), 2.20 (s, 3H), 2.37 (m, 2H), 3.95 (s, 3H), 6.84 (d, 1H), 7.65 (m, 2H).

### Step c) Preparation of 2-Amino-5-hexahydro-2,5-methanopentalen-3a(1H)-yl-5-(4-methoxy-3-methylphenyl)-3-methyl-3,5-dihydro-4H-imidazol-4-one

Using essentially the same procedure described in Example 7, step d, and employing 1-(hexahydro-2,5-methano-pentalen-3a-yl)-2-(4-methoxy-3-methyl-phenyl)ethane -1,2-dione (0.11 g, 0.36 mmol) and methylguanidine hydrochloride, the title product was obtained as a white solid (0.07 g, 54% yield, mp 160° C.); MS m/e (M+H)⁺ 354; ¹H NMR (400 MHZ, DMSO-d₆) δ 1.10-1.40 (m, 11H), 1.80 (m, 1H), 2.00 (m, 1H), 2.10 (s, 3H), 2.81 (s, 3H), 3.70 (s, 3H), 6.30 (bs, 2H), 6.78 (d, 1H), 7.42 (m, 2H).

### EXAMPLE 15

### Preparation of 5-(1-Adamantyl)-2-amino-3-methyl-5-(4′-methyl-1,1′-biphenyl-3-yl)-3,5-dihydro-4H-imidazol-4-one

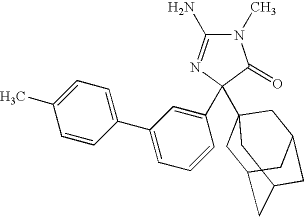

### Step a) Preparation of 1-(1-Adamantyl)-2-(3-bromophenyl)ethanone

3-Bromobenzyl bromide 1 (7.50 g) was dissolved in diethyl ether. The solution was cooled to 0-30° C. and magnesium (0.72 g) was added. The reaction mixture was stirred for 2 hours. Copper Bromide (I) (4.32 g) and anhydrous lithium bromide (5.22 g) were dissolved in tetrahydrofuran. The solution was cooled to −78° C. The Grignard solution was slowly added and it was kept at −78° C. A solution of adamantane-1-carbonyl chloride (5.94 g) in tetrahydrofuran was added and the reaction was stirred for 10 minutes at −78° C. and 10 minutes at 0° C. The reaction solution was diluted with diethyl ether and washed with 1 M hydrochloric acid and 1 M sodium hydroxide. The organic phase was dried over anhydrous magnesium sulfate and evaporated. The residue was purified by flash chromatography (hexanes:ethyl acetate 9:1) to afford the title ethanone compound as a colorless oil (5.70 g). ¹H NMR: 7.40-7.00 (m, 4H), 3.70 (s, 2H), 2.05 (s, 2H), 1.85 (s, 6H), 2.00-1.60 (m, 6H).

### Step b) Preparation of 5-(1-Adamantyl)-2-amino-3-methyl-5-(4′-methyl-1,1′-biphenyl-3-yl)-3,5-dihydro-4H-imidazol-4-one

A solution of 1-(1-adamantyl)-2-(3-bromophenyl)ethanone (121 mg) in DMF was treated with 4-methylphenylboronic acid (122 mg) followed by anhydrous potassium carbonate (250 mg) and tetrakis(triphenylphosphine) palladium (0) (35 mg). The reaction mixture was sealed, degassed and heated in a microwave oven at 150° C. for 30 h. The reaction was cooled and filtered. The filtrate was evaporated in vacuo. The resultant residue was dissolved in diethyl ether, washed with water, dried over anhydrous magnesium sulfate and evaporated to dryness. The resultant oil was dissolved in 1,4-dioxane, treated with selenium oxide (IV) (60 mg), heated at 95° C. overnight, cooled to room temperature, diluted with hexane and filtered. The filtrate was concentrated to a yellow oil residue. This oil was dissolved in ethyl alcohol, treated with 1-methylguanidine hydrochloride (42 mg) followed by a solution of sodium carbonate (119 mg) in water, heated at 70° C. overnight and concentrated in vacuo. The resultant residue was dissolved in DMSO and filtered. The filtrate was purified by Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD, to afford the title product as a white amorphous solid, characterized by LCMS analysis. LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD.

### EXAMPLES 16-26

### Preparation of 5-(1-Adamantyl)-2-amino-3-methyl-5-(substituted-phenyl)-3,5-dihydro-4H-imidazol-4-one Compounds

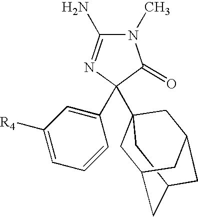

Using essentially the same procedure described in Example 15 hereinabove, the compounds shown in Table I were obtained and identified by HNMR and mass spectral analyses. LCMS conditions: HP 1100 HPLC system; Waters Xterra MS C18,2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD. In Table I, RT designates retention time.

TABLE I

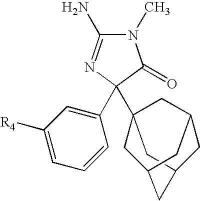

| Ex. | | | RT |
|---|---|---|---|
| No. | R4 | [M + H] | (min) |
| 16 | 4-methoxyphenyl | 430 | 2.75 |
| 17 | 3-methylphenyl | 414 | 2.87 |
| 18 | 3-methoxyphenyl | 430 | 2.74 |
| 19 | 3,4-dimethylphenyl | 428 | 3.06 |
| 20 | 3-cyanophenyl | 425 | 2.66 |
| 21 | 3-furylphenyl | 390 | 2.52 |
| 22 | 4-cyanophenyl | 425 | 2.67 |
| 23 | 3-(trifluoromethyl)phenyl | 468 | 3.01 |
| 24 | 3,4-difluorophenyl | 436 | 2.83 |
| 25 | phenyl | 400 | 2.77 |
| 26 | 2-methylphenyl | 414 | 2.82 |

### EXAMPLE 27

### Preparation of 5-(1-Adamantyl)-2-amino-5-(4-methoxyphenyl)-3-methyl-3,5-dihydro-4H-imidazol-4-one

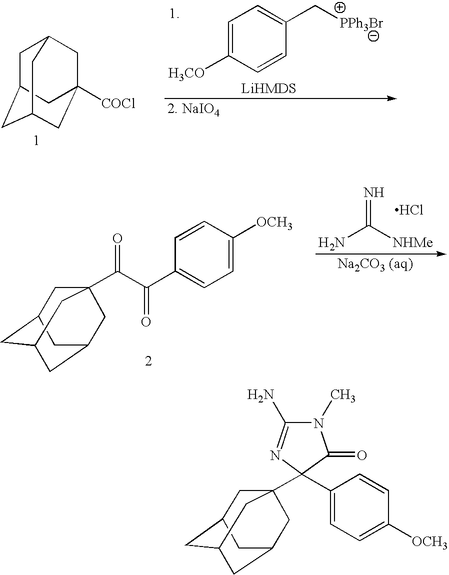

### Step a) Preparation of compound 2

A 1.0 M solution of lithium bis(trimethylsilyl)amide in tetrahydrofuran (52.3 mL, 52.3 mmol) was added to a stirred suspension of (4-methoxy-benzyl)-triphenylphosphonium chloride (21.9 g, 52.3 mmol) in tetrahydrofuran. The mixture was stirred for 15 min at room temperature, cooled to −5° C., treated with a solution of 1-admantanecarboxylic acid chloride 1 (9.44 g, 47.5 mmol) in THF and stirred for an additional 2 h while slowly warming to room temperature. The mixture was treated with water and sodium periodate (11.18 g, 52.3 mmol), stirred at 50° C. for 17 h, cooled to room temperature and diluted with ethyl acetate. The organic phase was separated and washed sequentially with water and brine, dried over sodium sulfate, filtered and concentrated. Purification of the resultant residue by flash chromatography (silica, 5:95 to 10:90 ethyl acetate/hexanes) afforded 2 (6.85 g, 48%) as a yellow solid: ¹H NMR (300 MHz, CDCl₃) δ 7.78 (d, J=8.9 Hz, 2H), 6.96 (d, J=8.9 Hz, 2H), 3.89 (s, 3H), 2.05-1.55 (m, 15H); ESI MS m/z 299 [C₁₉H₂₂O₃+H]⁺.

### Step b) Preparation of compound 5-(1-adamantyl)-2-amino-5-(4-methoxy-phenyl)-3-methyl-3,5-dihydro-4H-imidazol-4-one

A mixture of 2 (6.71 g, 22.5 mmol) and 1-methylguanidine hydrochloride (11.1 g, 101 mmol) in dioxane and ethanol was stirred at room temperature for 5 min, treated with an aqueous solution of sodium carbonate (10.7 g, 101 mmol), heated at 85° C. with stirring for 3.25 h, cooled to room temperature and concentrated in vacuo. Purification of the resultant residue by flash chromatography (silica, 95:5:0.5 methylene chloride/methanol/concentrated ammonium hydroxide) afforded the title product as a white solid, 3.41 g (43% yield), mp 150-155° C.; ¹H NMR (300 MHz, CDCl₃) δ 7.55 (d, J=6.9 Hz, 2H), 6.86 (d, J=6.9 Hz, 2H), 3.78 (s, 3H), 3.02 (s, 3H), 1.93-1.44 (m, 15H); IR (ATR) 3358, 2903, 1663, 1508, 1459, 1308, 1248, 1177, 1102, 1033, 998, 837, 804, 729 cm⁻¹; ESI MS m/z 354 [C₂₁H₂₇N₃O₂+H]⁺.

### EXAMPLE 28

### Preparation of (5S)-5-(1-Adamantyl)-2-amino-5-(4-methoxyphenyl)-3-methyl-3,5-dihydro-4H-imidazol-4-one [A] and (5R)-5-(1-Adamantyl)-2-amino-5-(4-methoxyphenyl)-3-methyl-3,5-dihydro-4H-imidazol-4-one [B]

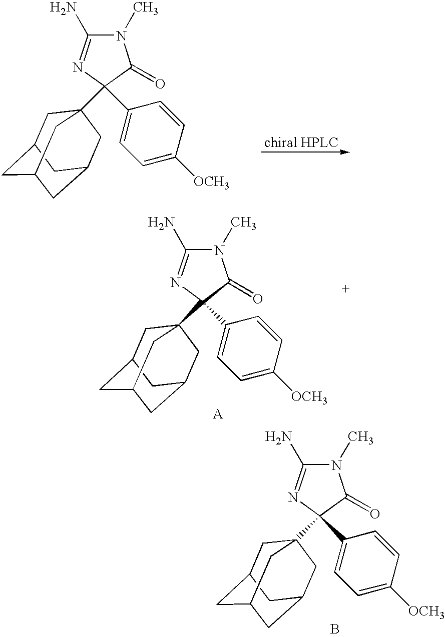

A racemic mixture of 5-(1-adamantyl)-2-amino-5-(4-methoxy-phenyl)-3-methyl-3,5-dihydro-4H-imidazol-4-one was separated to its two enantiomers by HPLC on Chiralcel AD, 0.46×25 cm using mobile phase EtOH:hexane (1:9 with 0.1% DEA) and a flow rate of 1.0 mL/min to afford the title S-isomer (A), mp 215° C.; [α]₂₅=−17.4 (C=1% in MeOH); ¹H NMR (400 MHZ, DMSO-d₆) δ 1.40 (m, 6H), 1.55 (m, 3H), 1.70 (m, 3H), 1.85 (bs,3H), 2.10 (s, 3H), 2.85 (s, 3H), 3.70(s, 3H), 6.40 (bs, 2H), 6.80 (d, 2H), 7.42 (d, 2H); MS m/e (M−H)-352; and the title R-isomer (B), mp 215° C.; [α]₂₅=−17.6 (C=1% in MeOH); ¹H NMR (400 MHZ, DMSO-d₆) δ 1.40 (m, 6H), 1.50 (m, 3H), 1.70 (m, 3H), 1.85 (bs,3H), 2.10 (s, 3H), 2.85 (s, 3H), 3.70 (s, 3H), 6.40 (bs, 2H), 6.80 (d, 2H), 7.42 (d, 2H); MS m/e (M−H)⁻ 352;

### EXAMPLE 29

### Preparation of 5(1-Adamantyl)-2-amino-3-methyl-5-[4-(trifluoromethoxy)phenyl]-3,5-dihydro-4H-imidazol-4-one

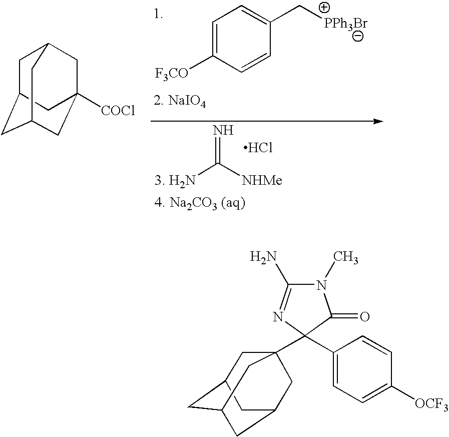

Using essentially the same procedure described in Example 27 and employing [4-(trifluoromethoxy)benzyl]triphenylphosphonium bromide in Step 1, the title compound was obtained as a white solid, mp 125° C., identified by NMR and mass spectral analyses.

### EXAMPLE 30

### Preparation of 5-[3-(benzyloxy)phenyl]-5-cyclohexyl-2-imino-3-methylimidazolidin-4-one

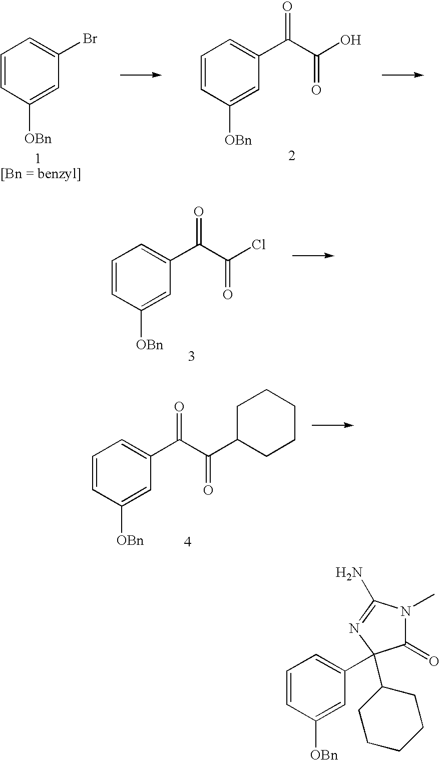

### Step a) Preparation of Compound 2

A mixture of 1 (11.84 g) and magnesium (1.62 g) in tetrahydrofuran (60 mL) was treated with 1,2-dibromoethane (100 μL), stirred at room temperature for 1 h, heated at reflux temperature for 1 h and allowed to cool to room temperature. In a separate flask, copper bromide (I) (6.84 g) and lithium bromide (7.83 g) were dissolved in tetrahydrofuran (50 mL). This solution was cooled to 0° C. and treated with the above Grignard solution. The reaction solution was slowly added, at −78° C., to oxalyl chloride (19.6 mL) in tetrahydrofuran (100 mL), held at −78° C. for 10 minutes, allowed to warm to room temperature over a 1 h period, carefully (heat evolution) poured into a flask containing brine (200 mL) and extracted with diethyl ether (300 mL). The organic phase was separated, washed with 1 M hydrochloric acid (200 mL) and extracted with 5M solution of sodium hydroxide. The basic aqueous extracts were combined, filtered through a Celite pad, acidified to pH 1 with concentrated hydrochloric acid, and extracted with diethyl ether. The ether extracts were combined, dried over magnesium sulfate and concentrated in vacuo to afford 2 as a yellow oil (6.67 g), by LCMS analysis (retention time 1.60 min, 255 [M−H]). LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD.

### Step b) Preparation of Compound 3

Compound (2) (6.67 g) was dissolved in dichloromethane, treated with DMF (500 μL), treated slowly with oxalyl chloride (26 mL) (gas evolution), stirred for 3 h and concentrated in vacuo. The resultant residue was dissolved in diethyl ether and filtered through Cellite. The filtrate was evaporated to dryness to afford 3 as a yellow liquid (5.94 g).

### Step c) Preparation of Compound 4

At 0° C., phenylmagnesium bromide (0.5 mL, 0.5 M solution in THF) was added to a solution of copper bromide (I) (111 mg) and lithium bromide (134 mg) in THF. This mixture was treated with a solution of acid chloride (3) (212 mg) in THF, allowed to warm to room temperature and diluted with diethyl ether, washed sequentially with 1 M hydrochloric acid and 1 M sodium hydroxide, dried over anhydrous magnesium sulfate and evaporated to dryness to give 4 as a yellow oil (200 mg).

### Step c) Preparation of 5-[3-(benzyloxy)phenyl]-5-cyclohexyl-2-imino-3-methylimidazolidin-4-one

A solution of 4 (200 mg) and 1-N-methylguanidine hydrochloride (85 mg) in ethanol, is treated with sodium carbonate (245 mg) in water, heated to 70° C. for 3 h and concentrated in vacuo. The resultant residue was purified using preparative reverse phase HPLC to give the title product as a white amphorous solid (18 mg), characterized by LCMS analysis, 348 [M+H]; Retention time 2.82 min. Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD. LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD.

### EXAMPLE 31

### Preparation of Adamantane-1-carbaldehyde

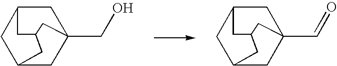

To a suspension of pyridinium chlorochromate (3.88 g, 18 mmol) in methylene chloride (100 ml) is added a solution of 1-adamantanemethanol (2.0 g, 12 mmol) in methylene chloride (30 ml) in one portion. After stirring for 2 h at room temperature, the reaction mixture is diluted with ether (50 ml). The mixture is filtered through a funnel packed with silica gel (20 g) and washed with ether. The filtrate is concentrated to afford the title compound 1.7 g (85%) as a white solid. mp: 132-135° C. MS (+) ES: 165 (M+H)⁺.

### EXAMPLE 32

### Preparation of 1-Ethynyladamantane

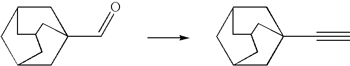

To a stirred mixture of adamantane-1-carbaldehyde (2.0 g, 12.2 mmol) and K₂CO₃ (3.36 g, 24.4 mmol) in methanol (200 ml) is added dimethyl (1-diazo-2-oxopropyl)phosphonate (2.8 g, 14.6 mmol) dropwise. After stirring for 4 h at room temperature, the reaction mixture is diluted with ether (100 ml). The mixture is washed with NaHCO₃ solution (5% in water, 300 ml). The organic layer is separated, dried (MgSO₄) and concentrated. The crude material is purified by chromatography (silica gel, 100% hexane) to afford the title compound 1.5 g (75%) as a white solid. mp: 80-82° C. MS (+) ES: 161 (M+H)⁺.

### EXAMPLE 33

### Preparation of 1-{[4-(Difluoromethoxy)phenyl]ethynyl}adamantine

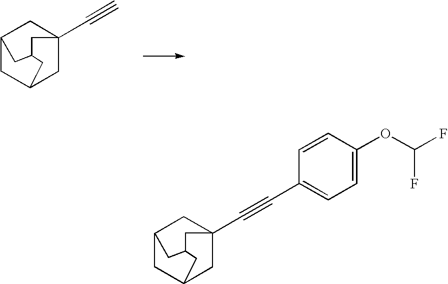

A mixture of 1-ethynyladamantane (320 mg, 2 mmol), 4-difluoromethoxy-4-iodobenzene (540 mg, 2 mmol), CuI (19 mg, 0.1 mmol) and Pd(PPh₃)₄ (92 mg, 0.08 mmol) in triethylamine (6 ml) and acetonitrile (3 ml) is refluxed for 3 h. After removal of the solvent, the crude mixture is purified by flash chromatography (silica gel, hexane/ethyl acetate: 95/5) to afford the title compound 470 mg (78%) as a clear oil. MS (+) ES: 303 (M+H)⁺.

### EXAMPLE 34

### Preparation of 1-(1-Adamantyl)-2-[4-(difluoromethoxy)phenyl]ethane-1,2-dione

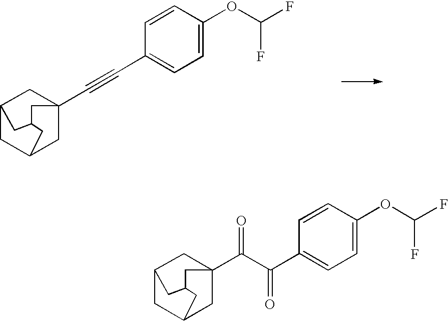

To a solution of 1-(4-difluoromethoxyphenylethynyl)adamantine (460 mg, 1.5 mmol) in acetone (20 ml) is added a solution of NaHCO₃ (77 mg, 0.91 mmol) and MgSO₄ (270 mg, 2.25 mmol) in water (10 ml), followed by the addition of KMnO₄ (711 mg, 4.5 mmol) in one portion. After stirring for 24 h at room temperature, the reaction mixture is extracted with hexane (2×20 ml). The combined organic extracts are dried (MgSO₄). Removal of the solvent affords the title compound 480 mg (94%) as a white solid. mp: 118-120° C. MS (+) ES: 335 (M+H)⁺.

### EXAMPLE 35

### Preparation of 5-(1-Adamantyl)-2-amino-5-[4-(difluoromethoxy)phenyl]-3-methyl-3,5-dihydro-4H-imidazol-4-one

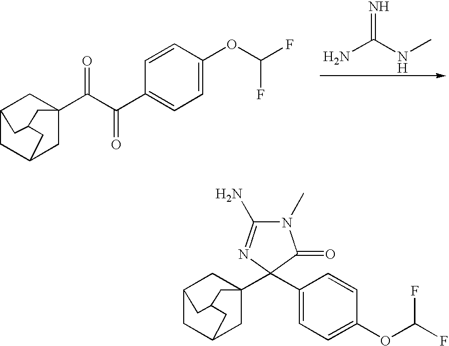

A mixture of 1-adamantan-1-yl-2-(4-difluoromethoxyphenylethynyl)ethane -1,2-dione (480 mg, 1.44 mmol), methylguanidine hydrochloride (313 mg, 2.88 mmol) and Na₂CO₃ (453 mg, 4.32 mmol) in ethanol and water is refluxed for 3 h and concentrated in vacuo. The resultant residue is purified by flash chromatography (silica gel, EtOAc/2.0 M ethanolic NH₃: 95/5) to afford the title compound 108 mg (20%) as a white solid, mp 216-218° C., identified by NMR and mass spectral analyses. MS (+) ES: 390 (M+H)⁺.

### EXAMPLE 36

### Preparation of 2-Amino-5-cyclohexyl-5-[4-(difluoromethoxy)phenyl]-3-methyl-3,5-dihydro-4H-imidazol-4-one

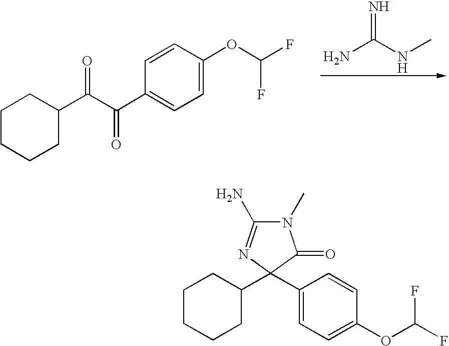

Using essentially the same procedure described in Example 35 and employing 1-cyclohexyl-2-(4-difluoromethoxyphenylethynyl)ethane-1,2-dione and methylguanidine, the title compound was obtained as a solid, mp 178-180° C., identified by NMR and mass spectral analyses. MS(+) APPI: 338 (M+H)⁺

### EXAMPLE 37

### Preparation of 2-Amino-5-cyclohexyl-3-(3,5-difluorobenzyl)-5-phenyl-3,5-dihydro-4H-imidazol-4-one

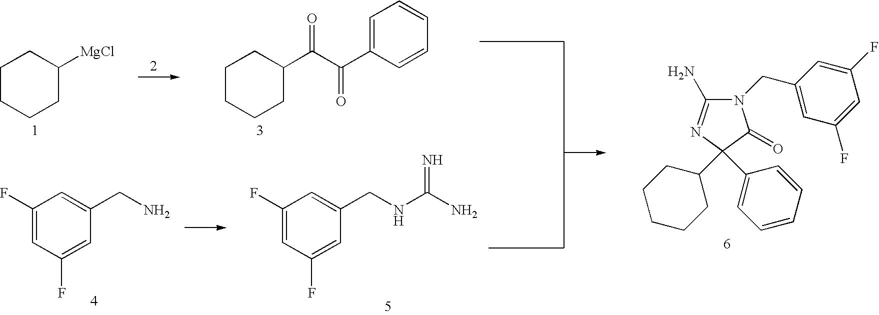

Phenylglycolic acid (2) (10.8 g) was dissolved in dichloromethane (200 mL). N,N-Dimethylformamide (100 uL) was added followed by slow addition of oxalyl chloride (72 mL, 2.0 M in dichloromethane) after 12 h the solvent was removed, and the residue was re-dissolved in diethyl ether (100 mL). The insoluble particles were filtered off (Celite) and the solvent was removed leaving brown liquid (11.80 g). ¹³C NMR: 181.1, 166.7, 135.9, 130.5, 129.4, 129.3

Copper bromide (I) (7.20 g) and lithium bromide (8.70 g) were dissolved in tetrahydrofuran (300 mL). The solution was cooled to −78° C. and cyclohexylmagnesium chloride (25 mL, 2.0 M in diethyl ether) was added. The solution was kept at this temperature for 10 minutes. Phenylglycolic acid chloride 2 (8.40 g) in tetrahydrofuran (10 mL) was added. The solution was stirred for 15 minutes and then it was warmed up to rt over 30 min. Diethyl ether (500 mL) was added and the solution was washed with 1 M hydrochloric acid (2×200 mL) and 1 M sodium hydroxide (200 mL). Drying with magnesium sulfate and removal of solvent produced crude product. Purification by flash chromatography (hexane:ethyl acetate 25:1) gave yellow oil (5.51 g). ¹H NMR: 8.00-8.90 (m, 2H), 7.70-58 (m, 1H), 7.56-7.40 (m, 2H), 3.18-3.00 (m, 1H), 2.00-1.05 (m, 10H). ¹³c NMR: 206.1, 194.2, 134.5, 132.5, 129.9, 128.8, 45.8, 27.1, 25.7, 25.3.

3,5-Difluorobenzylamine (72 mg) and 1-H-pyrazole-1-carboxamidine hydrochloride (73 mg) were dissolved in N,N-dimethylformamide. Diisopropylethylamine (0.74 mL) was added and the solution was heated at 40° C. overnight. The solvent was evaporated and the crude product was used without further purification.

Compound 3 (107.5 mg) and crude compound 5 were dissolved in ethanol (5 mL). Sodium carbonate (79 mg) in water (1 ml) was added and the solution was heated at 70° C. overnight. The solvent was evaporated and the crude product was purified using preparative reverse phase HPLC (Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD) providing white amphorous solid (32 mg). The compound was characterized by LCMS analysis (LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD.)

### EXAMPLES 38-73

### Preparation of 2-Amino-5-cyclohexyl-5-phenyl-3-substituted-3,5-dihydro-4H-imidazol-4-one Compounds

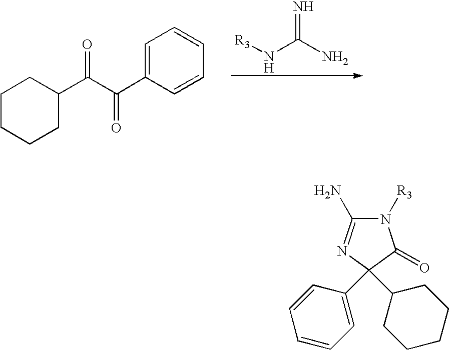

Using essentially the same procedure described in Example 37 and employing the appropriately substituted guanidine, the compounds shown in Table II were obtained and identified by HNMR and mass spectral analyses. LCMS conditions were the same as that used in Example 37.

TABLE II

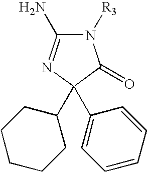

| Ex. | | | RT |
|---|---|---|---|
| No. | R3 | [M + H] | [min] |
| 38 | ethyl | 286 | 2.58 |
| 39 | propyl | 300 | 2.65 |
| 40 | 3-hydroxypropyl | 316 | 2.3 |
| 41 | 2,2,-diethoxyethyl | 374 | 2.64 |
| 42 | 2-phenylethyl | 362 | 2.50 |
| 43 | methyl | 272 | 2.30 |
| 44 | tetrahydrofuran-2-ylmethyl | 342 | 2.59 |
| 45 | 2-fluoroethyl | 304 | 2.45 |
| 46 | 2-difluoromethyloxybenzyl | 414 | 2.57 |
| 47 | N-acetyl-L-aspartyl | 431 | 2.32 |
| 48 | N-acetyl-D-aspartyl | 431 | 2.32 |
| 49 | 4-methylenecyclohexyl carboxylic acid | 398 | 1.95 |
| 50 | hexanoic acid | 372 | 2.46 |
| 51 | pentanoic acid | 358 | 1.89 |
| 52 | butanoin acid | 345 | 2.40 |
| 53 | 5-hydroxypropyl | 344 | 1.90 |
| 54 | propanoic acid | 330 | 2.40 |
| 55 | 1-methyl-3-propanoic acid | 344 | 1.88 |
| 56 | benzyl | 348 | 2.66 |
| 57 | 2-methylpropyl | 314 | 2.63 |
| 58 | hexyl | 342 | 2.93 |
| 59 | cyclohexyl | 340 | 2.71 |
| 60 | 2-hydroxybutyl | 330 | 2.36 |
| 61 | ethanecarboxylic acid | 316 | 2.37 |
| 62 | methylcyclohexyl | 354 | 2.31 |
| 63 | 2-furylmethyl | 338 | 2.40 |
| 64 | 4-hydroxyphenyl | 350 | 2.30 |
| 65 | 3-hydroxyphenyl | 350 | 2.30 |
| 66 | thien-2-ylmethyl | 354 | 2.40 |
| 67 | 4-methoxyphenyl | 364 | 3.30 |
| 68 | 2-thien-2-ylethyl | 368 | 3.30 |
| 69 | 2-(4-hydroxyphenyl)ethyl | 378 | 3.00 |
| 70 | 4-phenylacetic acid | 392 | 2.20 |
| 71 | 4-methylbenzoic acid | 392 | 2.40 |
| 72 | 5-(2-hydroxybenzoic acid) | 392 | 2.50 |
| 73 | ethyl-3-benzoate | 406 | 2.80 |

### EXAMPLES 74-88

### Preparation of 2-Amino-5-cycloalkyl-5-phenyl-3-substituted-3,5-dihydro-4H-imidazol -4-one Compounds

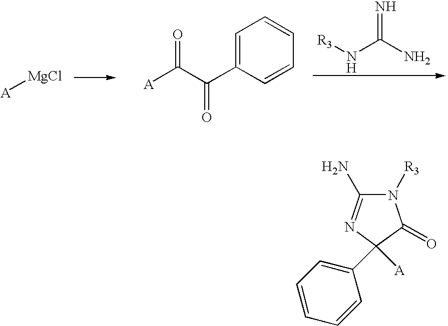

Using essentially the same procedure described in Example 37 and employing the appropriate cycloalkylmagnesium chloride and desired subtituted guanidine, the compounds shown in Table III were obtained and identified by HNMR and mass spectral analyses. The LCMS conditions used were the same as that described in Example 37. RT designates retention time.

TABLE III

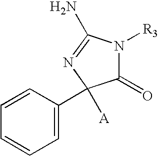

| Ex. | | | | RT |
|---|---|---|---|---|
| No. | R3 | A | [M + H] | (min) |
| 74 | methyl | cyclobutyl | 244 | 2.21 |
| 75 | methyl | 2-adamantyl | 324 | 2.83 |
| 76 | methyl | cyclopentyl | 258 | 2.34 |
| 77 | ethyl | cyclobutyl | 258 | 2.31 |
| 78 | ethyl | cycloheptyl | 300 | 2.63 |
| 79 | ethyl | 2-adamantyl | 338 | 2.55 |
| 80 | ethyl | cyclopentyl | 272 | 2.43 |
| 81 | propyl | cyclobutyl | 272 | 2.41 |
| 82 | propyl | cycloheptyl | 314 | 2.73 |
| 83 | propyl | 2-adamantyl | 352 | 3.01 |
| 84 | propyl | cyclopentyl | 286 | 2.51 |
| 85 | 3-hydroxypropyl | cyclobutyl | 288 | 2.08 |
| 86 | 3-hydroxypropyl | cycloheptyl | 330 | 2.42 |
| 87 | 3-hydroxypropyl | 2-adamantyl | 368 | 2.64 |
| 88 | 3-hydroxypropyl | cyclopentyl | 302 | 2.21 |

### EXAMPLE 89

### Preparation of 2-Amino-5-cyclohexyl-5-(2-methylphenyl)-3-methyl-3,5-dihydro -4H-imidazol-4-one

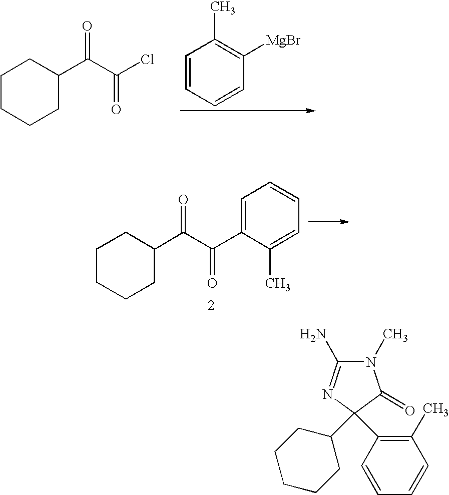

A solution of copper bromide (I) (72 mg) and lithium bromide (87 mg) in THF was cooled to 0° C., treated with 2-toluylmagnesium bromide (1 mL, 0.5 M in tetrahydrofuran), held at 0° C. for 10 minutes, treated with a solution of cyclohexylglycolic acid chloride (85 mg) in THF, stirred for 15 minutes, diluted with diethyl ether, washed sequentially with 1 M hydrochloric acid and 1 M sodium hydroxide, dried over magnesium sulfate and concentrated in vacuo to afford the diketone 2 as yellow oil (73 mg).

An ethanol solution of 2 (73 mg) and 1-N-methylguanidine hydrochloride (55 mg) was treated with sodium carbonate (159 mg) in water, heated at 70° C. overnight and evaporated to dryness. The resultant residue was purified by preparative HPLC (Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD) to give the title product as an amphorous solid (7 mg), [M+H] 286, retention time 1.73 min using LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD).

### EXAMPLES 90-107

### Preparation of 2-Amino-5-aryl-5-cyclohexyl-3-methyl-3,5-dihydro-4H-imidazol-4-one Compounds

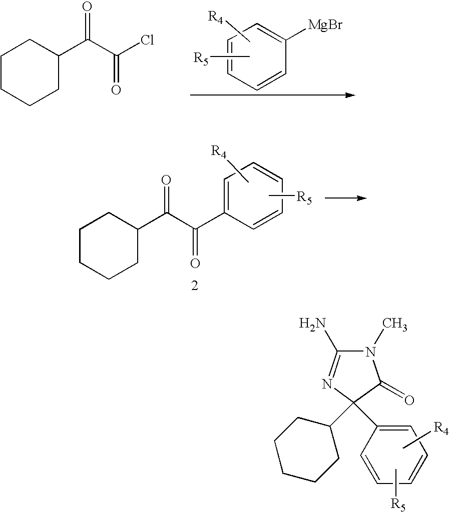

Using essentially the same procedure described in Example 89 and employing the appropriate phenylmagnesium bromide, the compounds shown in Table IV were obtained and identified by HNMR and mass spectral analyses. LCMS conditions are the same as those used in Example 89. RT designates retention time.

TABLE IV

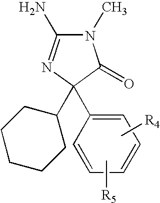

| Ex. | | | | RT |
|---|---|---|---|---|
| No. | R4 | R5 | [M + H] | [min] |
| 90 | 3-benzyl | H | 362 | 2.88 |
| 91 | 3-methyl | H | 286 | 1.83 |
| 92 | 4-methyl | H | 286 | 1.75 |
| 93 | 4-fluoro | H | 290 | 1.71 |
| 94 | 3-methoxy | H | 302 | 1.74 |
| 95 | 3-chloro | 4-chloro | 341 | 1.86 |
| 96 | 4-phenoxy | H | 364 | 1.93 |
| 97 | 3-chloro | H | 306 | 2.55 |
| 98 | 3-chloro | 5-chloro | 341 | 1.85 |
| 99 | 2-phenyl | H | 348 | 1.9 |
| 100 | 4-phenyl | H | 348 | 1.99 |
| 101 | 2-methyl | 5-methyl | 300 | 1.81 |
| 102 | 4-trifluoromethyl | H | 340 | 1.86 |
| 103 | 2-methoxy | H | 302 | 1.78 |
| 104 | 4-methoxy | H | 302 | 1.74 |
| 105 | 4-chloro | H | 306 | 1.86 |

| | | | |
|---|---|---|---|
| 106 | 2-CH═CH—CH═CH-3 | 322 | 1.82 |
| 107 | 3-CH═CH—CH═CH-4 | 322 | 1.85 |

### EXAMPLE 108

### Preparation of 2-Amino-5-cyclohexyl-5-(3-cyclopentylphenyl)-3-methyl-3,5-dihydro-4H-imidazol-4-one

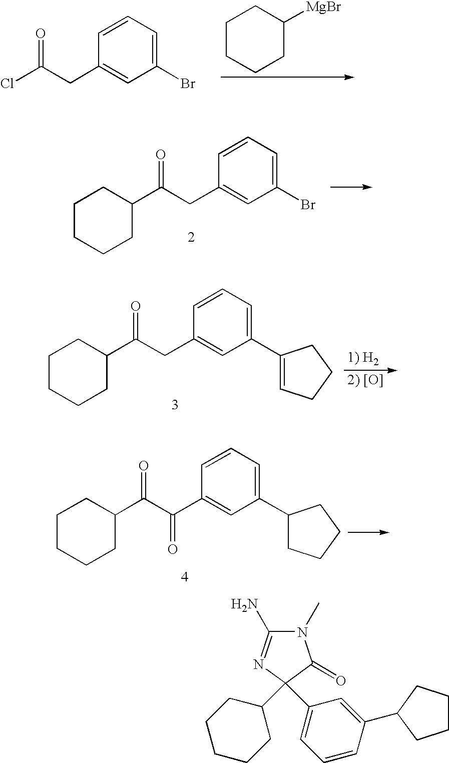

A solution of copper bromide (I) (2.47 g) and anhydrous lithium bromide (2.99 g) in THF at −78° C. was treated with cyclohexylmagnesium chloride (8.59 mL, 2.0 M solution in diethyl ether) followed by a solution of 3-bromophenylacetyl chloride (4.00 g) in THF, stirred for 10 minutes, allowed to come to room temperature, diluted with diethyl ether, washed sequentially with 1 M hydrochloric acid and 1 M sodium hydroxide, dried over anhydrous magnesium sulfate and evaporated. The resultant residue was taken up in hexane:ethyl acetate 4:1 and filtered through a pad of silica gel to give 2 as a colorless oil (4.61 g); ¹H NMR: 7.40-7.05 (m, 4H), 3.70 (s, 2H), 2.40 (m, 1H), 1.90-1.60 (m, 5H), 1.40-1.05 (m, 5H); ¹³C NMR: 210.2, 136.5, 132.4, 129.9, 129.8, 128.1, 122.4, 50.4, 46.9, 28.4, 25.7, 25.5.

A solution of 2 (280 mg) in DMF (1.0 mL) was treated with diisopropylethylamine (1.0 mL) followed by palladium acetate (II) (23 mg), tri(o-tollyl)phosphine (61 mg) and cyclopentene (0.5 mL). The reaction mixture was heated in a microwave oven for 300 seconds at 150° C. and evaporated to dryness. The residue was taken in diethyl ether, washed with water, dried over magnesium sulfate and concentrated in vacuo to give a dark oil residue. The oil was purified by preparative reverse phase HPLC system¹ to give compound 3.
¹ Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD) system and removal of solvents provided.

A solution of 3 in methanol was treated with palladium hydroxide (20 mg, 10% on carbon) and hydrogenated at atmospheric pressure for 6 hours. The resultant reaction mixture was filtered and the filtrate was evaporated to dryness to give a brown residue. This residue was dissolved in dioxane, treated with Selenium dioxide (IV) (100 mg), heated at 95° C. overnight, cooled to ambient temperatures, diluted with hexanes and filtered. The filtrate was concentrated in vacuo to give the diketone 4 as a brown oil (ca 70 mg).

Diketone 4 was reacted with methyl guanidine in essentially the same manner described in Example 27, Step b, to give the title product, after purification by Gilson preparative reverse phase HPLC¹ as a white amphorous solid (93 mg), characterized by LCMS analysis², 340[M+H], retention time 2.65 minutes.
² LCMS Conditions: HP 1100 HPLC system; Waters Xterra MS C18, 2 mm (i.d.)×50 mm (length), 3.5 um column, set at 50° C.; Flow rate 1.0 mL/min; Solvent A: 0.02% NH₄OH in water; Solvent B 0.02% NH₄OH in ACN; Gradient: Time O: 10% B; 2.5 min 90% B; 3 min 90% B; Sample concentration: ˜2.0 mM; Injection volume: 5 uL; Detection: 220 nm, 254 nm DAD)

### EXAMPLE 109

### Preparation of 2-Amino-5-cyclohexyl-5-(3-cyclohexylphenyl)-3-methyl-3,5-dihydro-4H-imidazol-4-one

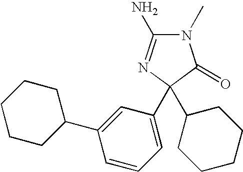

Using essentially the same procedure described in Example 108 and employing cyclohexene in the Heck coupling reaction, the title product was obtained, LCMS* 2.65 min., [M+H} 340.
* Conditions were the same as those used in Example 108.

### EXAMPLE 110

### Preparation of N-[3-(2-amino-4-cyclohexyl-1-methyl-5-oxo-4,5-dihydro-1H-imidazol-4-yl)phenyl]-3-methoxybenzamide

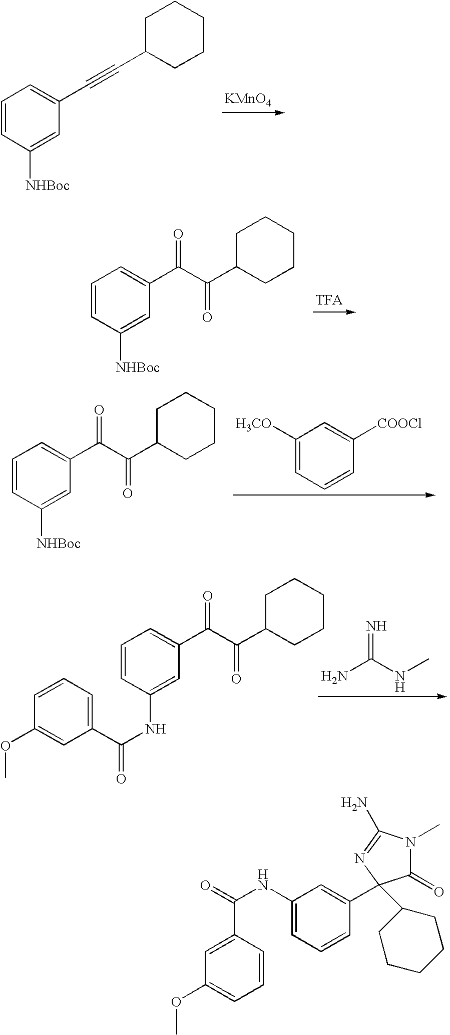

Using essentially the same procedures described in Examples 34 and 35 and employing 3-methoxybenzoylchloride the title product was obtained, and identified using the Gilson preparative reverse phase HPLC system: YMC Pro C18, 20 mm×50 mm ID, 5 uM column; 2 mL injection; Solvent A: 0.02% NH₄OH/water; Solvent B: 0.02% NH₄OH/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD, retention time 2.6 min., 420 [M+H].

### EXAMPLES 111-135

### Preparation of N-[3-(2-Amino-4-cyclohexyl-1-methyl-5-oxo-4,5-dihydro-1H-imidazol-4-yl)phenyl]-benzamide Derivatives

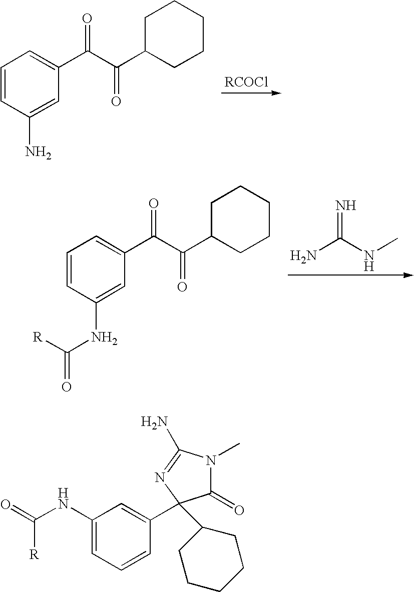

Using essentially the same procedure described in Example 110 and employing a suitable acid chloride, the compounds shown in Table V were obtained and identified by LC and mass spectral analyses. (LCMS conditions are the same as those used in Example 110.

TABLE V

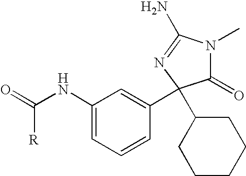

| Ex. | | | RT |
|---|---|---|---|
| No. | R | [M + H] | [min] |
| 111 | 3-methoxyphenyl | 421 | 2.6 |
| 112 | 2-methoxyethyl | 373 | 2.29 |
| 113 | methoxyphenylmethyl | 435 | 2.63 |
| 114 | 3-methyl-2-furyl | 395 | 2.63 |
| 115 | (2-methoxyethoxy)methyl | 403 | 2.36 |
| 116 | (N,N-dimethylamino)methyl | | |
| 117 | 3-(N,N-dimethylamino)phenyl | 434 | 2.74 |
| 118 | 3-(N,N-dimethylamino)propyl | 400 | 1.47 |
| 119 | 4-(1-methylpiperidyl) | 412 | 1.47 |
| 120 | methylcyclopropyl | 369 | 2.44 |
| 121 | (1-phenoxy-1-methyl)methyl | 435 | 1.99 |
| 122 | 3-(trifluoromethyl)-phenyl | 459 | 2.03 |
| 123 | 2-methoxybenzyl | 435 | 2.61 |
| 124 | 2-N-methylpyrroyl | 394 | 2.58 |
| 125 | methoxymethyl | 359 | 2.28 |
| 126 | 2-furanyl | 381 | 2.43 |
| 127 | benzyloxymethyl | 435 | 2.68 |
| 128 | 2-methoxyphenyl | 421 | 2.75 |
| 129 | 3,4-dimethoxyphenyl | 451 | 2.56 |
| 130 | 2,5-dimethoxyphenyl | 465 | 2.61 |
| 131 | 1-E-propenyl | 355 | 2.42 |
| 132 | propyl | 357 | 2.44 |
| 133 | 3-methoxybenzyl | 435 | 2.57 |
| 134 | 5-(1,3benzo[1.3]dioxoyl) | 435 | 2.57 |
| 135 | 2-(2-(chlorophenoxy)propane) | 484 | 2.13 |

### EXAMPLE 136

### Preparation of (5S)-5-(1-Adamantyl)-2-amino-5-(4-difluoromethoxyphenyl)-3-methyl-3,5-dihydro-4H-imidazol-4-one Trifluoroacetic Acid Salt [A] and (5R)-5-(1-Adamantyl)-2-amino-5-(4-difluoromethoxyphenyl)-3-methyl-3,5-dihydro-4H-Trifluoroacetic Acid Salt [B]

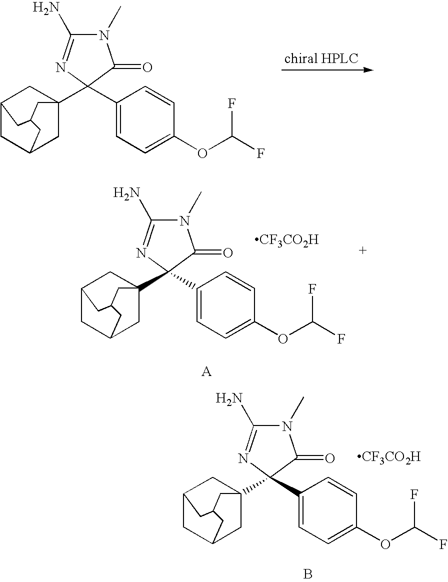

A racemic mixture of 5-(1-adamantyl)-2-amino-5-(4-difluoromethoxyphenyl)-3-methyl-3,5-dihydro-4H-imidazol-4-one was separated by HPLC on Chiralcel AD-H, 25×2 cm using mobile phase 10% (20% ethanol in hexane/TFA) in hexane/TFA and a flow rate of 21 mL/min to afford the title S-isomer (A), mp 225-226° C.; [α]₂₅=+10.4 (C=1% in DMSO); ¹H NMR (400 MHZ, DMSO-d₆) 61.40 (t, 6H), 1.55 (q, 6H), 1.92 (s, 3H), 3.06 (s, 3H), 7.22 (d, 2H), 7.22 (t, 1H), 7.58 (d, 2H); MS m/e (M−H)⁻ 388; and the title R-isomer (B), mp 225-226° C.; [α]₂₅=−12.8 (C=1% in DMSO); ¹H NMR (400 MHZ, DMSO-d₆) δ 1.40 (t, 6H), 1.55 (q, 6H), 1.92 (s, 3H), 3.06 (s, 3H), 7.22 (d, 2H), 7.22 (t, 1H), 7.58 (d, 2H); MS m/e (M−H)⁻ 388.

### EXAMPLE 137

Evaluation of BACE-1 Binding Affinity of Test Compounds

Fluorescent Kinetic Assay

Final Assay Conditions: 10 nM human BACE1 (or 10 nM Murine BACE1), 25 μM substrate (WABC-6, MW 1549.6, from AnaSpec), Buffer: 50 mM Na-Acetate, pH 4.5, 0.05% CHAPS, 25% PBS, room temperature. Na-Acetate was from Aldrich, Cat.# 24, 124-5, CHAPS was from Research Organics, Cat. # 1304C 1×, PBS was from Mediatech (Cellgro), Cat# 21-031-CV, peptide substrate AbzSEVNLDAEFRDpa was from AnaSpec, Peptide Name: WABC-6

Determination of stock substrate (AbzSEVNLDAEFRDpa) concentration: ˜25 mM stock solution is made in DMSO using the peptide weight and MW, and diluted to ˜25 μM (1:1000) in 1×PBS. Concentration is determined by absorbance at 354 nm using an extinction coefficient ε of 18172 M⁻¹ cm⁻¹, the concentration of stock substrate is corrected, and the substrate stock stored in small aliquots in −80° C. [Substrate Stock]=ABS^{354 nm}*10⁶/18172 (in mM)

The extinction coefficient ε^{354 nm} was adapted from TACE peptide substrate, which had the same quencher-fluorophore pair.

Determination of Stock Enzyme Concentration: the stock concentration of each enzyme is determined by absorbance at 280 nm using ε of 64150 M⁻¹ cm⁻¹ for hBACE1 and MuBACE1 in 6 M Guanidinium Hydrochloride (from Research Organics, Cat. # 5134G-2), pH ˜6. The extinction coefficient ε^{280 nm} for each enzyme was calculated based on known amino acid composition and published extinction coefficients for Trp (5.69 M⁻¹ cm⁻¹) and Tyr (1.28 M⁻¹ cm⁻¹) residues (Anal. Biochem. 182, 319-326).

Dilution and mixing steps: total reaction volume: 100 μL

2× inhibitor dilutions in buffer A (66.7 mM Na-Acetate, pH 4.5, 0.0667% CHAPS) were prepared,

4× enzyme dilution in buffer A (66.7 mM Na-Acetate, pH 4.5, 0.0667% CHAPS) were prepared,

100 μM substrate dilution in 1×PBS was prepared, and

50 μL 2× Inhibitor, 25 μL 100 μM substrate are added to each well of 96-well plate (from DYNEX Technologies, VWR #: 11311-046), immediately followed by 25 μL 4× enzyme (added to the inhibitor and substrate mix), and the fluorescence readings are initiated.

Fluorescence Readings: Readings at λₑₓ 320 nm and λₑₘ 420 nm are taken every 40 sec for 30 min at room temperature and the linear slope for substrate cleavage rate (vᵢ) determined.

Calculation of % Inhibition:

% Inhibition=100*(1−vᵢ/v₀)

vᵢ: substrate cleavage rate in the presence of inhibitor

v₀: substrate cleavage rate in the absence of inhibitor

IC₅₀ Determination:

% Inhibition=((B*IC₅₀ⁿ)+(100*I₀ⁿ))/(IC₅₀ⁿ+I₀ⁿ)

(Model # 39 from LSW Tool Bar in Excel where B is the % inhibition from the enzyme control, which should be close to 0.) % Inhibition is plotted vs. Inhibitor Concentration (I₀) and the data fit to the above equation to obtain IC₅₀ value and Hill number (n) for each compound. Testing at least 10 different inhibitor concentrations is preferred. The data obtained are shown in Table VI below.

TABLE VI

| Ex. | BACE 1 |
|---|---|
| No. | IC₅₀ μM |
| 1 | A |
| 2 | A |
| 3 | A |
| 4 | A |
| 5 | A |
| 6 | A |
| 7 | B |
| 8 | A |
| 9 | A |
| 10 | C |
| 11 | A |
| 12 | B |
| 13 | B |
| 14 | A |
| 17 | C |
| 22 | C |
| 25 | C |
| 26 | C |
| 27 | A |
| 28 | A |
| 29 | A |
| 30 | C |
| 36 | C |
| 44 | A |
| 46 | A |
| 64 | A |
| 67 | A |
| 72 | A |
| 90 | B |
| 92 | B |
| 108 | A |
| 107 | A |
| 114 | A |
| 124 | A |
| 125 | A |
| 126 | A |
| 135 | A |
| 136A | C |
| 136B | C |

For Table VI
A = 0.01 μM-1.00 μM
B = 1.10 μM-10.0 μM
C = >10.0 μM

What is claimed is:

1. A compound of formula I 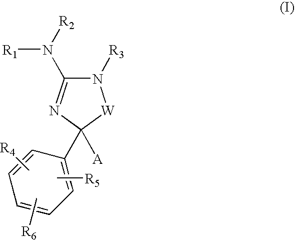
wherein
A is cycloalkyl;
W is CO, CS or CH₂;
R₁, R₂, and R₃ are each independently H, or an alkyl, cycloalkyl, cycloheteroalkyl, aryl or heteroaryl group, each group optionally substituted, or R₁ and R₂ may be taken together with the atom to which they are attached to form an optionally substituted 5- to 7-membered ring optionally interrupted by an additional heteroatom selected from O, N or S;
R₄, R₅, and R₆ are each independently H, halogen, NO₂, CN, OR₇, COR₇, CO₂R₇, CONR₈R₉, NR₈R₉, NR₈COR₇, NR₈SO₂R₁₀, SO₂NR₈R₉ or SOₙR₁₀ or an alkyl, alkenyl, alkynyl, cycloalkyl, cycloheteroalkyl, aryl, or heteroaryl group, each group optionally substituted, or when attached to adjacent carbon atoms R₄ and R₅ or R₅ and R₆ may be taken together with the atoms to which they are attached to form a an optionally substituted 5- to 7-membered ring optionally interrupted by one, two or three heteroatoms selected from O, N or S;
n is 0, 1, or 2;
R₇ is independently at each occurrence H, or an alkyl, alkenyl, alkynyl, cycloalkyl, cycloheteroalkyl, aryl, or heteroaryl group each group optionally substituted;
R₈ and R₉ are each independently at each occurrence H, OR₇, COR₇, CO₂R₇ or an alkyl, alkenyl, alkynyl, cycloalkyl, cycloheteroalkyl, aryl, or heteroaryl group, each group optionally substituted, or R₈ and R₉ may be taken together with the atom to which they are attached to form an optionally substituted 5- to 7-membered ring optionally interrupted by an additional heteroatom selected from O, N or S; and
R₁₀ is independently at each occurrence an alkyl, alkenyl, alkynyl, cycloalkyl, cycloheteroalkyl, aryl or heteroaryl group each group optionally substituted; or
a tautomer thereof, a stereoisomer thereof or a pharmaceutically acceptable salt thereof.

2. The compound according to claim 1 wherein W is CO.

3. The compound according to claim 1 wherein A is adamantyl.

4. The compound according to claim 1 wherein R₁ and R₂ are H and R₃ is C₁-C₄ alkyl.

5. The compound according to claim 1 wherein R₅ is OR₇.

6. The compound according to claim 2 wherein A is adamantyl and R₁ and R₂ are H.

7. The compound according to claim 2 wherein R₃ is C₁-C₄ alkyl and R₅ is OR₇.

8. The compound according to claim 7 wherein R₅ is OR₇ and R₇ is CHF₂.

9. The compound according to claim 1 selected from the group consisting essentially of:
(5S)-5-(1-adamantyl)-2-amino-5-[4-(difluoromethoxy)phenyl]-3-methyl-3,5-dihydro-4H-imidazol-4-one;
2-amino-5-bicyclo[2.2.1]hept-1-yl-5-(4-methoxy-3-methylphenyl)-3-methyl-3,5-dihydro-4H-imidazol-4-one;
5-(1-adamantyl)-2-amino-5-(4-methoxyphenyl)-3-methyl-3,5-dihydro-4H-imidazol-4-one;
5-(1-adamantyl)-2-amino-5-(4-ethoxyphenyl)-3-methyl-3,5-dihydro-4H-imidazol-4-one;
5-(1-adamantyl)-2-amino-5-(4-butoxyphenyl)-3-methyl-3,5-dihydro-4H-imidazol-4-one;
5-(1-adamantyl)-2-amino-5-(3-ethyl-4-methoxyphenyl)-3-methyl-3,5-dihydro-4H-imidazol -4-one;
5-(1-adamantyl)-2-amino-5-(4-methoxy-3,5-dimethylphenyl)-3-methyl-3,5-dihydro-4H-imidazol -4-one;
5-(1-adamantyl)-2-amino-3-methyl-5-phenyl-3,5-dihydro-4H-imidazol-4-one;
5-(1-adamantyl)-2-amino-5-(4-methoxy-3-methylphenyl)-3-methyl-3,5-dihydro-4H-imidazol -4-one;
(5S)-5-(1-adamantyl)-2-amino-5-(4-methoxy-3-methylphenyl)-3-methyl-3,5-dihydro -4H-imidazol-4-one;
(5R)-5-(1-adamantyl)-2-amino-5-(4-methoxy-3-methylphenyl)-3-methyl-3,5-dihydro -4H-imidazol-4-one;
5-(1-adamantyl)-2-amino-5-(3,4-dimethoxyphenyl)-3-methyl-3,5-dihydro-4H-imidazol -4-one;
5-(1-adamantyl)-2-amino-5-(4-methoxy-2,3-dimethyl phenyl)-3-methyl-3,5-dihydro-4H-imidazol -4-one;
2-amino-5-bicyclo[2.2.1]hept-2-yl-5-(4-methoxy-3-methylphenyl)-3-methyl-3,5-dihydro-4H-imidazol-4-one;
2-amino-5-hexahydro-2,5-methanopentalen-3a(1H)-yl-5-(4-methoxy-3-methylphenyl)-3-methyl-3,5-dihydro-4H-imidazol-4-one;
5-(1-adamantyl)-2-amino-3-methyl-5-(4′-methyl-1,1′-biphenyl-3-yl)-3,5-dihydro-4H-imidazol-4-one;
5-(1-adamantyl)-2-amino-5-(4′-methoxy-1,1′-biphenyl-3-yl)-3-methyl-3,5-dihydro-4H-imidazol-4-one;
5-(1-adamantyl)-2-amino-3-methyl-5-(3′-methyl-1,1′-biphenyl-3-yl)-3,5-dihydro-4H-imidazol-4-one;
5-(1-adamantyl)-2-amino-5-(3′-methoxy-1,1′-biphenyl-3-yl)-3-methyl-3,5-dihydro-4H-imidazol-4-one;
5-(1-adamantyl)-2-amino-5-(3′,4′-dimethyl-1,1′-biphenyl-3-yl)-3-methyl-3,5-dihydro -4H-imidazol-4-one;
3′-[4-(1-adamantyl)-2-amino-1-methyl-5-oxo-4,5-dihydro-1H-imidazol-4-yl]-1,1′-biphenyl-3-carbonitrile;
5-(1-adamantyl)-2-amino-5-[3-(3-furyl)phenyl]-3-methyl-3,5-dihydro-4H-imidazol-4-one;
3′-[4-(1-adamantyl)-2-amino-1-methyl-5-oxo-4,5-dihydro-1H-imidazol-4-yl]-1,1′-biphenyl-4-carbonitrile;
3′-[4-(1-adamantyl)-2-amino-1-methyl-5-oxo-4,5-dihydro-1H-imidazol-4-yl]-1,1′-biphenyl-4-carbonitrile;
5-(1-adamantyl)-2-amino-5-(3′,4′-difluoro-1,1′-biphenyl-3-yl)-3-methyl-3,5-dihydro-4H-imidazol -4-one;
5-(1-adamantyl)-2-amino-5-(1,1′-biphenyl-3-yl)-3-methyl-3,5-dihydro-4H-imidazol-4-one;
5-(1-adamantyl)-2-amino-3-methyl-5-(2′-methyl-1,1′-biphenyl-3-yl)-3,5-dihydro-4H-imidazol -4-one;
2-amino-5-cyclohexyl-3-(3,5-difluorobenzyl)-5-phenyl-3,5-dihydro-4H-imidazol-4-one;
5-cyclohexyl-3-ethyl-2-imino-5-phenylimidazolidin-4-one;
5-cyclohexyl-2-imino-5-phenyl-3-propylimidazolidin-4-one;
5-cyclohexyl-3-(3-hydroxypropyl)-2-imino-5-phenylimidazolidin-4-one;
2-amino-5-cyclohexyl-3-(2,2-diethoxyethyl)-5-phenyl-3,5-dihydro-4H-imidazol-4-one;
2-amino-5-cyclohexyl-5-phenyl-3-(2-phenylethyl)-3,5-dihydro-4H-imidazol-4-one;
5-cyclohexyl-2-imino-3-methyl-5-phenylimidazolidin-4-one;
2-amino-5-cyclohexyl-5-phenyl-3-(tetrahydrofuran-2-ylmethyl)-3,5-dihydro-4H-imidazol -4-one;
2-amino-5-cyclohexyl-3-(2-fluoroethyl)-5-phenyl-3,5-dihydro-4H-imidazol-4-one;
2-amino-5-cyclohexyl-3-[2-(difluoromethoxy)benzyl]-5-phenyl-3,5-dihydro-4H-imidazol -4-one;
N-[(2-amino-4-cyclohexyl-5-oxo-4-phenyl-4,5-dihydro-1H-imidazol-1-yl)acetyl]-L-aspartic acid;
N-[(2-amino-4-cyclohexyl-5-oxo-4-phenyl-4,5-dihydro-1H-imidazol-1-yl)acetyl]-D-aspartic acid;
trans-4-[(2-amino-4-cyclohexyl-5-oxo-4-phenyl-4,5-dihydro-1H-imidazol-1-yl)methyl]cyclohexanecarboxylic acid;
6-(2-amino-4-cyclohexyl-5-oxo-4-phenyl-4,5-dihydro-1H-imidazol-1-yl)hexanoic acid;
5-(2-amino-4-cyclohexyl-5-oxo-4-phenyl-4,5-dihydro-1H-imidazol-1-yl)pentanoic acid;
4-(2-amino-4-cyclohexyl-5-oxo-4-phenyl-4,5-dihydro-1H-imidazol-1-yl)butanoic acid;
2-amino-5-cyclohexyl-3-(5-hydroxypentyl)-5-phenyl-3,5-dihydro-4H-imidazol-4-one;
3-(2-amino-4-cyclohexyl-5-oxo-4-phenyl-4,5-dihydro-1H-imidazol-1-yl)propanoic acid;
3-(2-amino-4-cyclohexyl-5-oxo-4-phenyl-4,5-dihydro-1H-imidazol-1-yl)-2-methylpropanoic acid;
2-amino-3-benzyl-5-cyclohexyl-5-phenyl-3,5-dihydro-4H-imidazol-4-one;
2-amino-5-cyclohexyl-3-isobutyl-5-phenyl-3,5-dihydro-4H-imidazol-4-one;
2-amino-5-cyclohexyl-3-hexyl-5-phenyl-3,5-dihydro-4H-imidazol-4-one;
2-amino-3,5-dicyclohexyl-5-phenyl-3,5-dihydro-4H-imidazol-4-one;
2-amino-5-cyclohexyl-3-(4-hydroxybutyl)-5-phenyl-3,5-dihydro-4H-imidazol-4-one;
(2-amino-4-cyclohexyl-5-oxo-4-phenyl-4,5-dihydro-1H-imidazol-1-yl)acetic acid;
2-amino-5-cyclohexyl-3-(cyclohexylmethyl)-5-phenyl-3,5-dihydro-4H-imidazol-4-one;
2-amino-5-cyclohexyl-3-(2-furylmethyl)-5-phenyl-3,5-dihydro-4H-imidazol-4-one;
2-amino-5-cyclohexyl-3-(4-hydroxyphenyl)-5-phenyl-3,5-dihydro-4H-imidazol-4-one;
2-amino-5-cyclohexyl-3-(3-hydroxyphenyl)-5-phenyl-3,5-dihydro-4H-imidazo-4-one;
2-amino-5-cyclohexyl-5-phenyl-3-(thien-2-ylmethyl)-3,5-dihydro-4H-imidazol-4-one;
2-amino-5-cyclohexyl-3-(4-methoxyphenyl)-5-phenyl-3,5-dihydro-4H-imidazo-4-one;
2-amino-5-cyclohexyl-5-phenyl-3-(2-thien-2-ylethyl)-3,5-dihydro-4H-imidazol-4-one;
2-amino-5-cyclohexyl-3-[2-(4-hydroxyphenyl)ethyl]-5-phenyl-3,5-dihydro-4H-imidazol -4-one;
[4-(2-amino-4-cyclohexyl-5-oxo-4-phenyl-4,5-dihydro-1H-imidazol-1-yl)phenyl]acetic acid;
4-[(2-amino-4-cyclohexyl-5-oxo-4-phenyl-4,5-dihydro-1H-imidazol-1-yl)methyl]benzoic acid;
5-(2-amino-4-cyclohexyl-5-oxo-4-phenyl-4,5-dihydro-1H-imidazol-1-yl)-2-hydroxybenzoic acid;
ethyl 3-(2-amino-4-cyclohexyl-5-oxo-4-phenyl-4,5-dihydro-1H-imidazol-1-yl)benzoate;
5-cyclobutyl-2-imino-3-methyl-5-phenylimidazolidin-4-one;
5-(2-adamantyl)-2-imino-3-methyl-5-phenylimidazolidin-4-one;
5-cyclopentyl-2-imino-3-methyl-5-phenylimidazolidin-4-one;
5-cyclobutyl-3-ethyl-2-imino-5-phenylimidazolidin-4-one;
5-cycloheptyl-3-ethyl-2-imino-5-phenylimidazolidin-4-one;
5-(2-adamantyl)-3-ethyl-2-imino-5-phenylimidazolidin-4-one;
5-cyclopentyl-3-ethyl-2-imino-5-phenylimidazolidin-4-one;
5-cyclobutyl-2-imino-5-phenyl-3-propylimidazolidin-4-one;
5-cycloheptyl-2-imino-5-phenyl-3-propylimidazolidin-4-one;
5-(2-adamantyl)-2-imino-5-phenyl-3-propylimidazolidin-4-one;
5-cyclopentyl-2-imino-5-phenyl-3-propylimidazolidin-4-one;
5-cyclobutyl-3-(3-hydroxypropyl)-2-imino-5-phenylimidazolidin-4-one;
5-cycloheptyl-3-(3-hydroxypropyl)-2-imino-5-phenylimidazolidin-4-one;
5-(2-adamantyl)-3-(3-hydroxypropyl)-2-imino-5-phenylimidazolidin-4-one;
5-cyclopentyl-3-(3-hydroxypropyl)-2-imino-5-phenylimidazolidin-4-one;
5-cyclohexyl-2-imino-3-methyl-5-(2-methylphenyl)imidazolidin-4-one;
5-(3-benzylphenyl)-5-cyclohexyl-2-imino-3-methylimidazolidin-4-one;
5-cyclohexyl-2-imino-3-methyl-5-(3-methylphenyl)imidazolidin-4-one;
5-cyclohexyl-2-imino-3-methyl-5-(4-methylphenyl)imidazolidin-4-one;
5-cyclohexyl-5-(4-fluorophenyl)-2-imino-3-methylimidazolidin-4-one;
5-cyclohexyl-2-imino-5-(3-methoxyphenyl)-3-methylimidazolidin-4-one;
5-cyclohexyl-5-(3,4-dichlorophenyl)-2-imino-3-methylimidazolidin-4-one;
5-cyclohexyl-2-imino-3-methyl-5-(4-phenoxyphenyl)imidazolidin-4-one;
5-(3-chlorophenyl)-5-cyclohexyl-2-imino-3-methylimidazolidin-4-one;
5-cyclohexyl-5-(3,5-dichlorophenyl)-2-imino-3-methylimidazolidin-4-one;
5-(1,1′-biphenyl-2-yl)-5-cyclohexyl-2-imino-3-methylimidazolidin-4-one;
5-(1,1′-biphenyl-4-yl)-5-cyclohexyl-2-imino-3-methylimidazolidin-4-one;
5-cyclohexyl-5-(2,5-dimethylphenyl)-2-imino-3-methylimidazolidin-4-one;
5-cyclohexyl-2-imino-3-methyl-5-[4-(trifluoromethyl)phenyl]imidazolidin-4-one;
5-cyclohexyl-2-imino-5-(2-methoxyphenyl)-3-methylimidazolidin-4-one;
5-cyclohexyl-2-imino-5-(4-methoxyphenyl)-3-methylimidazolidin-4-one;
5-(4-chlorophenyl)-5-cyclohexyl-2-imino-3-methylimidazolidin-4-one;
2-amino-5-cyclohexyl-5-(3-cyclopentylphenyl)-3-methyl-3,5-dihydro-4H-imidazol-4-one;
2-amino-5-cyclohexyl-5-(3-cyclohexylphenyl)-3-methyl-3,5-dihydro-4H-imidazol-4-one;
5-[3-(benzyloxy)phenyl]-5-cyclohexyl-2-imino-3-methylimidazolidin-4-one;
N-{3-[2-amino-4-(4-methoxy-3-methylphenyl)-1-methyl-5-oxo-4,5-dihydro-1H-imidazol -4-yl]phenyl}-2-(4-chlorophenoxy)-2-methylpropanamide;
N-[3-(2-amino-4-cyclohexyl-1-methyl-5-oxo-4,5-dihydro-1H-imidazol-4-yl)phenyl]-3-methoxybenzamide;
N-[3-(2-amino-4-cyclohexyl-1-methyl-5-oxo-4,5-dihydro-1H-imidazol-4-yl)phenyl]-3-methoxypropanamide;
(2R)-N-[3-(2-amino-4-cyclohexyl-1-methyl-5-oxo-4,5-dihydro-1H-imidazol-4-yl)phenyl]-2-methoxy-2-phenylacetamide;
N-[3-(2-amino-4-cyclohexyl-1-methyl-5-oxo-4,5-dihydro-1H-imidazol-4-yl)phenyl]-3-methyl-2-furamide;
N-[3-(2-amino-4-cyclohexyl-1-methyl-5-oxo-4,5-dihydro-1H-imidazol-4-yl)phenyl]-2-(2-methoxyethoxy)acetamide;
N˜1˜-[3-(2-amino-4-cyclohexyl-1-methyl-5-oxo-4,5-dihydro-1H-imidazol-4-yl)phenyl]-N˜2˜,N˜2˜-dimethylglycinamide;
N-[3-(2-amino-4-cyclohexyl-1-methyl-5-oxo-4,5-dihydro-1H-imidazol-4-yl)phenyl]-3-(dimethylamino)benzamide;
N-[3-(2-amino-4-cyclohexyl-1-methyl-5-oxo-4,5-dihydro-1H-imidazol-4-yl)phenyl]-4-(dimethylamino)butanamide;
N-[3-(2-amino-4-cyclohexyl-1-methyl-5-oxo-4,5-dihydro-1H-imidazol-4-yl)phenyl]-1-methylpiperidine-4-carboxamide;
N-[3-(2-amino-4-cyclohexyl-1-methyl-5-oxo-4,5-dihydro-1H-imidazol-4-yl)phenyl]-2-cyclopropylacetamide;
N-[3-(2-amino-4-cyclohexyl-1-methyl-5-oxo-4,5-dihydro-1H-imidazol-4-yl)phenyl]-2-phenoxypropanamide;
N-[3-(2-amino-4-cyclohexyl-1-methyl-5-oxo-4,5-dihydro-1H-imidazol-4-yl)phenyl]-3-(trifluoromethyl)benzamide;
N-[3-(2-amino-4-cyclohexyl-1-methyl-5-oxo-4,5-dihydro-1H-imidazol-4-yl)phenyl]-2-(2-methoxyphenyl)acetamide;
N-[3-(2-amino-4-cyclohexyl-1-methyl-5-oxo-4,5-dihydro-1H-imidazol-4-yl)phenyl]-1-methyl-1H-pyrrole-2-carboxamide;
N-[3-(2-amino-4-cyclohexyl-1-methyl-5-oxo-4,5-dihydro-1H-imidazol-4-yl)phenyl]-2-methoxyacetamide;
N-[3-(2-amino-4-cyclohexyl-1-methyl-5-oxo-4,5-dihydro-1H-imidazol-4-yl)phenyl]-2-furamide;
N-[3-(2-amino-4-cyclohexyl-1-methyl-5-oxo-4,5-dihydro-1H-imidazol-4-yl)phenyl]-2-(benzyloxy)acetamide;
N-[3-(2-amino-4-cyclohexyl-1-methyl-5-oxo-4,5-dihydro-1H-imidazol-4-yl)phenyl]-2-methoxybenzamide;
N-[3-(2-amino-4-cyclohexyl-1-methyl-5-oxo-4,5-dihydro-1H-imidazol-4-yl)phenyl]-3,4-dimethoxybenzamide;
N-[3-(2-amino-4-cyclohexyl-1-methyl-5-oxo-4,5-dihydro-1H-imidazol-4-yl)phenyl]-2-(2,5-dimethoxyphenyl)acetamide;
(2E)-N-[3-(2-amino-4-cyclohexyl-1-methyl-5-oxo-4,5-dihydro-1H-imidazol-4-yl)phenyl]but-2-enamide;
N-[3-(2-amino-4-cyclohexyl-1-methyl-5-oxo-4,5-dihydro-1H-imidazol-4-yl)phenyl]butanamide;
N-[3-(2-amino-4-cyclohexyl-1-methyl-5-oxo-4,5-dihydro-1H-imidazol-4-yl)phenyl]-2-(3-methoxyphenyl)acetamide;
N-[3-(2-amino-4-cyclohexyl-1-methyl-5-oxo-4,5-dihydro-1H-imidazol-4-yl)phenyl]-1,3-benzodioxole-5-carboxamide;
N-[3-(2-amino-4-cyclohexyl-1-methyl-5-oxo-4,5-dihydro-1H-imidazol-4-yl)phenyl]-2-(4-chlorophenoxy)-2-methylpropanamide;
a tautomer thereof;
a stereoisomer thereof; and
a pharmaceutically acceptable salt thereof.

10. A method for the treatment of a disease or disorder associated with excessive BACE activity in a patient in need thereof which comprises providing to said patient a therapeutically effective amount of a compound of formula I 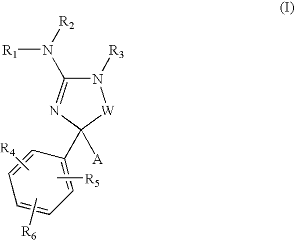
wherein
A is cycloalkyl;
W is CO, CS or CH₂;
R₁, R₂, and R₃ are each independently H, or an alkyl, cycloalkyl, cycloheteroalkyl, aryl or heteroaryl group, each group optionally substituted, or R₁ and R₂ may be taken together with the atom to which they are attached to form an optionally substituted 5- to 7-membered ring optionally interrupted by an additional heteroatom selected from O, N or S;
R₄, R₅, and R₆ are each independently H, halogen, NO₂, CN, OR₇, COR₇, CO₂R₇, CONR₈R₉, NR₈R₉, NR₈COR₇, NR₈SO₂R₁₀, SO₂NR₈R₉ or SOₙR₁₀ or an alkyl, alkenyl, alkynyl, cycloalkyl, cycloheteroalkyl, aryl, or heteroaryl group, each group optionally substituted, or when attached to adjacent carbon atoms R₄ and R₅ or R₅ and R₆ may be taken together with the atoms to which they are attached to form a an optionally substituted 5- to 7-membered ring optionally interrupted by one, two or three heteroatoms selected from O, N or S;
n is 0, 1, or 2;
R₇ is independently at each occurrence H, or an alkyl, alkenyl, alkynyl, cycloalkyl, cycloheteroalkyl, aryl, or heteroaryl group each group optionally substituted;
R₈ and R₉ are each independently at each occurrence H, OR₇, COR₇, CO₂R₇ or an alkyl, alkenyl, alkynyl, cycloalkyl, cycloheteroalkyl, aryl, or heteroaryl group, each group optionally substituted, or R₈ and R₉ may be taken together with the atom to which they are attached to form an optionally substituted 5- to 7-membered ring optionally interrupted by an additional heteroatom selected from O, N or S; and
R₁₀ is independently at each occurrence an alkyl, alkenyl, alkynyl, cycloalkyl, cycloheteroalkyl, aryl or heteroaryl group each group optionally substituted; or
a tautomer thereof, a stereoisomer thereof or a pharmaceutically acceptable salt thereof.

11. The method according to claim 10 wherein said disease or disorder is selected from the group consisting essentially of: Alzheimer's disease; cognitive impairment; Down's Syndrome; HCHWA-D; cognitive decline; senile dementia; cerebral amyloid angiopathy; and a neurodegenerative disorder.

12. The method according to claim 10 wherein said disease or disorder is characterized by the production of β-amyloid deposits or neurofibrillary tangles.

13. The method according to claim 11 wherein said disease or disorder is Alzheimer's disease.

14. A method for modulating the activity of BACE which comprises contacting a receptor thereof with an effective amount of a compound according to claim 1.

15. A pharmaceutical composition which comprises a pharmaceutically acceptable carrier and an effective amount of a compound of formula 1 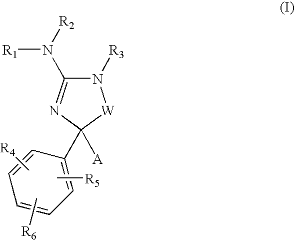
wherein
A is cycloalkyl;
W is CO, CS or CH₂;
R₁, R₂, and R₃ are each independently H, or an alkyl, cycloalkyl, cycloheteroalkyl, aryl or heteroaryl group, each group optionally substituted, or R₁ and R₂ may be taken together with the atom to which they are attached to form an optionally substituted 5- to 7-membered ring optionally interrupted by an additional heteroatom selected from O, N or S;
R₄, R₅, and R₆ are each independently H, halogen, NO₂, CN, OR₇, COR₇, CO₂R₇, CONR₈R₉, NR₈R₉, NR₈COR₇, NR₈SO₂R₁₀, SO₂NR₈R₉ or SOₙR₁₀ or an alkyl, alkenyl, alkynyl, cycloalkyl, cycloheteroalkyl, aryl, or heteroaryl group, each group optionally substituted, or when attached to adjacent carbon atoms R₄ and R₅ or R₅ and R₆ may be taken together with the atoms to which they are attached to form a an optionally substituted 5- to 7-membered ring optionally interrupted by one, two or three heteroatoms selected from O, N or S;
n is 0, 1, or 2;
R₇ is independently at each occurrence H, or an alkyl, alkenyl, alkynyl, cycloalkyl, cycloheteroalkyl, aryl, or heteroaryl group each group optionally substituted;
R₈ and R₉ are each independently at each occurrence H, OR₇, COR₇, CO₂R₇ or an alkyl, alkenyl, alkynyl, cycloalkyl, cycloheteroalkyl, aryl, or heteroaryl group, each group optionally substituted, or R₈ and R₉ may be taken together with the atom to which they are attached to form an optionally substituted 5- to 7-membered ring optionally interrupted by an additional heteroatom selected from O, N or S; and
R₁₀ is independently at each occurrence an alkyl, alkenyl, alkynyl, cycloalkyl, cycloheteroalkyl, aryl or heteroaryl group each group optionally substituted; or
a tautomer thereof, a stereoisomer thereof or a pharmaceutically acceptable salt thereof.

16. The composition according to claim 15 having a formula I compound wherein W is CO.

17. The composition according to claim 16 having a formula I compound wherein A is adamantyl and R₁ and R₂ are H.

18. The composition according to claim 17 having a formula I compound wherein R₃ is methyl.

19. The composition according to claim 18 having a formula I compound wherein R₅ is OR₇.

20. The composition according to claim 15 having a formula I compound selected from the group consisting essentially of:
(5S)-5-(1-adamantyl)-2-amino-5-[4-(difluoromethoxy)phenyl]-3-methyl-3,5-dihydro -4H-imidazol-4-one;
2-amino-5-bicyclo[2.2.1]hept-1-yl-5-(4-methoxy-3-methylphenyl)-3-methyl-3,5-dihydro-4H-imidazol-4-one;
5-(1-adamantyl)-2-amino-5-(4-methoxyphenyl)-3-methyl-3,5-dihydro-4H-imidazol-4-one;
5-(1-adamantyl)-2-amino-5-(4-ethoxyphenyl)-3-methyl-3,5-dihydro-4H-imidazol-4-one;
5-(1-adamantyl)-2-amino-5-(4-butoxyphenyl)-3-methyl-3,5-dihydro-4H-imidazol-4-one;
5-(1-adamantyl)-2-amino-5-(3-ethyl-4-methoxyphenyl)-3-methyl-3,5-dihydro-4H-imidazol -4-one;
5-(1-adamantyl)-2-amino-5-(4-methoxy-3,5-dimethylphenyl)-3-methyl-3,5-dihydro-4H-imidazol -4-one;
5-(1-adamantyl)-2-amino-3-methyl-5-phenyl-3,5-dihydro-4H-imidazol-4-one;
5-(1-adamantyl)-2-amino-5-(4-methoxy-3-methylphenyl)-3-methyl-3,5-dihydro-4H-imidazol -4-one;
(5S)-5-(1-adamantyl)-2-amino-5-(4-methoxy-3-methylphenyl)-3-methyl-3,5-dihydro -4H-imidazol-4-one;
(5R)-5-(1-adamantyl)-2-amino-5-(4-methoxy-3-methylphenyl)-3-methyl-3,5-dihydro -4H-imidazol-4-one;
5-(1-adamantyl)-2-amino-5-(3,4-dimethoxyphenyl)-3-methyl-3,5-dihydro-4H-imidazol -4-one;
5-(1-adamantyl)-2-amino-5-(4-methoxy-2,3-dimethylphenyl)-3-methyl-3,5-dihydro-4H-imidazol -4-one;
2-amino-5-bicyclo[2.2.1]hept-2-yl-5-(4-methoxy-3-methylphenyl)-3-methyl-3,5-dihydro-4H-imidazol-4-one;
2-amino-5-hexahydro-2,5-methanopentalen-3a(1H)-yl-5-(4-methoxy-3-methylphenyl) -3-methyl-3,5-dihydro-4H-imidazol-4-one;
5-(1-adamantyl)-2-amino-3-methyl-5-(4′-methyl-1,1′-biphenyl-3-yl)-3,5-dihydro-4H-imidazol -4-one;
5-(1-adamantyl)-2-amino-5-(4′-methoxy-1,1′-biphenyl-3-yl)-3-methyl-3,5-dihydro-4H-imidazol -4-one;
5-(1-adamantyl)-2-amino-3-methyl-5-(3′-methyl-1,1′-biphenyl-3-yl)-3,5-dihydro-4H-imidazol -4-one;
5-(1-adamantyl)-2-amino-5-(3′-methoxy-1,1′-biphenyl-3-yl)-3-methyl-3,5-dihydro-4H-imidazol -4-one;
5-(1-adamantyl)-2-amino-5-(3′,4′-dimethyl-1,1′-biphenyl-3-yl)-3-methyl-3,5-dihydro -4H-imidazol-4-one;
3′-[4-(1-adamantyl)-2-amino-1-methyl-5-oxo-4,5-dihydro-1H-imidazol-4-yl]-1,1′-biphenyl-3-carbonitrile;
5-(1-adamantyl)-2-amino-5-[3-(3-furyl)phenyl]-3-methyl-3,5-dihydro-4H-imidazo-4-one;
3′-[4-(1-adamantyl)-2-amino-1-methyl-5-oxo-4,5-dihydro-1H-imidazol-4-yl]-1,1′biphenyl-4-carbonitrile;
3′-[4-(1-adamantyl)-2-amino-1-methyl-5-oxo-4,5-dihydro-1H-imidazol-4-yl]-1,1′-biphenyl-4-carbonitrile;
5-(1-adamantyl)-2-amino-5-(3′,4′-difluoro-1,1′-biphenyl-3-yl)-3-methyl-3,5-dihydro-4H-imidazol -4-one;
5-(1-adamantyl)-2-amino-5-(1,1′-biphenyl-3-yl)-3-methyl-3,5-dihydro-4H-imidazol-4-one;
5-(1-adamantyl)-2-amino-3-methyl-5-(2′-methyl-1,1′-biphenyl-3-yl)-3,5-dihydro-4H-imidazol -4-one;
2-amino-5-cyclohexyl-3-(3,5-difluorobenzyl)-5-phenyl-3,5-dihydro-4H-imidazol-4-one;
5-cyclohexyl-3-ethyl-2-imino-5-phenylimidazolidin-4-one;
5-cyclohexyl-2-imino-5-phenyl-3-propylimidazolidin-4-one;
5-cyclohexyl-3-(3-hydroxypropyl)-2-imino-5-phenylimidazolidin-4-one;
2-amino-5-cyclohexyl-3-(2,2-diethoxyethyl)-5-phenyl-3,5-dihydro-4H-imidazol-4-one;
2-amino-5-cyclohexyl-5-phenyl-3-(2-phenylethyl)-3,5-dihydro-4H-imidazol-4-one;
5-cyclohexyl-2-imino-3-methyl-5-phenylimidazolidin-4-one;
2-amino-5-cyclohexyl-5-phenyl-3-(tetrahydrofuran-2-ylmethyl)-3,5-dihydro-4H-imidazol-4-one;
2-amino-5-cyclohexyl-3-(2-fluoroethyl)-5-phenyl-3,5-dihydro-4H-imidazol-4-one;
2-amino-5-cyclohexyl-3-[2-(difluoromethoxy)benzyl]-5-phenyl-3,5-dihydro-4H-imidazol -4-one;
N-[(2-amino-4-cyclohexyl-5-oxo-4-phenyl-4,5-dihydro-1H-imidazol-1-yl)acetyl]-L-aspartic acid;
N-[(2-amino-4-cyclohexyl-5-oxo-4-phenyl-4,5-dihydro-1H-imidazol-1-yl)acetyl]-D-aspartic acid;
trans-4-[(2-amino-4-cyclohexyl-5-oxo-4-phenyl-4,5-dihydro-1H-imidazol-1-yl)methyl]cyclohexanecarboxylic acid;
6-(2-amino-4-cyclohexyl-5-oxo-4-phenyl-4,5-dihydro-1H-imidazol-1-yl)hexanoic acid;
5-(2-amino-4-cyclohexyl-5-oxo-4-phenyl-4,5-dihydro-1H-imidazol-1-yl)pentanoic acid;
4-(2-amino-4-cyclohexyl-5-oxo-4-phenyl-4,5-dihydro-1H-imidazol-1-yl)butanoic acid;
2-amino-5-cyclohexyl-3-(5-hydroxypentyl)-5-phenyl-3,5-dihydro-4H-imidazol-4-one;
3-(2-amino-4-cyclohexyl-5-oxo-4-phenyl-4,5-dihydro-1H-imidazol-1-yl)propanoic acid;
3-(2-amino-4-cyclohexyl-5-oxo-4-phenyl-4,5-dihydro-1H-imidazol-1-yl)-2-methylpropanoic acid;
2-amino-3-benzyl-5-cyclohexyl-5-phenyl-3,5-dihydro-4H-imidazol-4-one;
2-amino-5-cyclohexyl-3-isobutyl-5-phenyl-3,5-dihydro-4H-imidazol-4-one;
2-amino-5-cyclohexyl-3-hexyl-5-phenyl-3,5-dihydro-4H-imidazol-4-one;
2-amino-3,5-dicyclohexyl-5-phenyl-3,5-dihydro-4H-imidazol-4-one;
2-amino-5-cyclohexyl-3-(4-hydroxybutyl)-5-phenyl-3,5-dihydro-4H-imidazol-4-one;
(2-amino-4-cyclohexyl-5-oxo-4-phenyl-4,5-dihydro-1H-imidazol-1-yl)acetic acid;
2-amino-5-cyclohexyl-3-(cyclohexylmethyl)-5-phenyl-3,5-dihydro-4H-imidazol-4-one;
2-amino-5-cyclohexyl-3-(2-furylmethyl)-5-phenyl-3,5-dihydro-4H-imidazol-4-one;
2-amino-5-cyclohexyl-3-(4-hydroxyphenyl)-5-phenyl-3,5-dihydro-4H-imidazol-4-one;
2-amino-5-cyclohexyl-3-(3-hydroxyphenyl)-5-phenyl-3,5-dihydro-4H-imidazol-4-one;
2-amino-5-cyclohexyl-5-phenyl-3-(thien-2-ylmethyl)-3,5-dihydro-4H-imidazol-4-one;
2-amino-5-cyclohexyl-3-(4-methoxyphenyl)-5-phenyl-3,5-dihydro-4H-imidazol-4-one;
2-amino-5-cyclohexyl-5-phenyl-3-(2-thien-2-ylethyl)-3,5-dihydro-4H-imidazol-4-one;
2-amino-5-cyclohexyl-3-[2-(4-hydroxyphenyl)ethyl]-5-phenyl-3,5-dihydro-4H-imidazol -4-one;
[4-(2-amino-4-cyclohexyl-5-oxo-4-phenyl-4,5-dihydro-1H-imidazol-1-yl)phenyl]acetic acid;
4-[(2-amino-4-cyclohexyl-5-oxo-4-phenyl-4,5-dihydro-1H-imidazol-1-yl)methyl]benzoic acid;
5-(2-amino-4-cyclohexyl-5-oxo-4-phenyl-4,5-dihydro-1H-imidazol-1-yl)-2-hydroxybenzoic acid;
ethyl 3-(2-amino-4-cyclohexyl-5-oxo-4-phenyl-4,5-dihydro-1H-imidazol-1-yl)benzoate;
5-cyclobutyl-2-imino-3-methyl-5-phenylimidazolidin-4-one;
5-(2-adamantyl)-2-imino-3-methyl-5-phenylimidazolidin-4-one;
5-cyclopentyl-2-imino-3-methyl-5-phenylimidazolidin-4-one;
5-cyclobutyl-3-ethyl-2-imino-5-phenylimidazolidin-4-one;
5-cycloheptyl-3-ethyl-2-imino-5-phenylimidazolidin-4-one;
5-(2-adamantyl)-3-ethyl-2-imino-5-phenylimidazolidin-4-one;
5-cyclopentyl-3-ethyl-2-imino-5-phenylimidazolidin-4-one;
5-cyclobutyl-2-imino-5-phenyl-3-propylimidazolidin-4-one;
5-cycloheptyl-2-imino-5-phenyl-3-propylimidazolidin-4-one;
5-(2-adamantyl)-2-imino-5-phenyl-3-propylimidazolidin-4-one;
5-cyclopentyl-2-imino-5-phenyl-3-propylimidazolidin-4-one;
5-cyclobutyl-3-(3-hydroxypropyl)-2-imino-5-phenylimidazolidin-4-one;
5-cycloheptyl-3-(3-hydroxypropyl)-2-imino-5-phenylimidazolidin-4-one;
5-(2-adamantyl)-3-(3-hydroxypropyl)-2-imino-5-phenylimidazolidin-4-one;
5-cyclopentyl-3-(3-hydroxypropyl)-2-imino-5-phenylimidazolidin-4-one;
5-cyclohexyl-2-imino-3-methyl-5-(2-methylphenyl)imidazolidin-4-one;
5-(3-benzylphenyl)-5-cyclohexyl-2-imino-3-methylimidazolidin-4-one;
5-cyclohexyl-2-imino-3-methyl-5-(3-methylphenyl)imidazolidin-4-one;
5-cyclohexyl-2-imino-3-methyl-5-(4-methylphenyl)imidazolidin-4-one;
5-cyclohexyl-5-(4-fluorophenyl)-2-imino-3-methylimidazolidin-4-one;
5-cyclohexyl-2-imino-5-(3-methoxyphenyl)-3-methylimidazolidin-4-one;
5-cyclohexyl-5-(3,4-dichlorophenyl)-2-imino-3-methylimidazolidin-4-one;
5-cyclohexyl-2-imino-3-methyl-5-(4-phenoxyphenyl)imidazolidin-4-one;
5-(3-chlorophenyl)-5-cyclohexyl-2-imino-3-methyl imidazolidin-4-one;
5-cyclohexyl-5-(3,5-dichlorophenyl)-2-imino-3-methylimidazolidin-4-one;
5-(1,1′-biphenyl-2-yl)-5-cyclohexyl-2-imino-3-methylimidazolidin-4-one;
5-(1,1′-biphenyl-4-yl)-5-cyclohexyl-2-imino-3-methylimidazolidin-4-one;
5-cyclohexyl-5-(2,5-dimethylphenyl)-2-imino-3-methylimidazolidin-4-one;
5-cyclohexyl-2-imino-3-methyl-5-[4-(trifluoromethyl)phenyl]imidazolidin-4-one;
5-cyclohexyl-2-imino-5-(2-methoxyphenyl)-3-methylimidazolidin-4-one;
5-cyclohexyl-2-imino-5-(4-methoxyphenyl)-3-methyl imidazolidin-4-one;
5-(4-chlorophenyl)-5-cyclohexyl-2-imino-3-methylimidazolidin-4-one;
2-amino-5-cyclohexyl-5-(3-cyclopentylphenyl)-3-methyl-3,5-dihydro-4H-imidazol-4-one;
2-amino-5-cyclohexyl-5-(3-cyclohexylphenyl)-3-methyl-3,5-dihydro-4H-imidazol-4-one;
5-[3-(benzyloxy)phenyl]-5-cyclohexyl-2-imino-3-methylimidazolidin-4-one;
N-{3-[2-amino-4-(4-methoxy-3-methylphenyl)-1-methyl-5-oxo-4,5-dihydro-1H-imidazol -4-yl]phenyl}-2-(4-chlorophenoxy)-2-methylpropanamide;
N-[3-(2-amino-4-cyclohexyl-1-methyl-5-oxo-4,5-dihydro-1H-imidazol-4-yl)phenyl]-3-methoxybenzamide;
N-[3-(2-amino-4-cyclohexyl-1-methyl-5-oxo-4,5-dihydro-1H-imidazol-4-yl)phenyl]-3-methoxypropanamide;
(2R)-N-[3-(2-amino-4-cyclohexyl-1-methyl-5-oxo-4,5-dihydro-1H-imidazol-4-yl)phenyl]-2-methoxy-2-phenylacetamide;
N-[3-(2-amino-4-cyclohexyl-1-methyl-5-oxo-4,5-dihydro-1H-imidazol-4-yl)phenyl]-3-methyl-2-furamide;
N-[3-(2-amino-4-cyclohexyl-1-methyl-5-oxo-4,5-dihydro-1H-imidazol-4-yl)phenyl]-2-(2-methoxyethoxy)acetamide;
N˜1˜-[3-(2-amino-4-cyclohexyl-1-methyl-5-oxo-4,5-dihydro-1H-imidazol-4-yl)phenyl]-N˜2˜,N˜2˜-dimethylglycinamide;
N-[3-(2-amino-4-cyclohexyl-1-methyl-5-oxo-4,5-dihydro-1H-imidazol-4-yl)phenyl]-3-(dimethylamino)benzamide;
N-[3-(2-amino-4-cyclohexyl-1-methyl-5-oxo-4,5-dihydro-1H-imidazol-4-yl)phenyl]-4-(dimethylamino)butanamide;
N-[3-(2-amino-4-cyclohexyl-1-methyl-5-oxo-4,5-dihydro-1H-imidazol-4-yl)phenyl]-1-methylpiperidine-4-carboxamide;
N-[3-(2-amino-4-cyclohexyl-1-methyl-5-oxo-4,5-dihydro-1H-imidazol-4-yl)phenyl]-2-cyclopropylacetamide;
N-[3-(2-amino-4-cyclohexyl-1-methyl-5-oxo-4,5-dihydro-1H-imidazol-4-yl)phenyl]-2-phenoxypropanamide;
N-[3-(2-amino-4-cyclohexyl-1-methyl-5-oxo-4,5-dihydro-1H-imidazol-4-yl)phenyl]-3-(trifluoromethyl)benzamide;
N-[3-(2-amino-4-cyclohexyl-1-methyl-5-oxo-4,5-dihydro-1H-imidazol-4-yl)phenyl]-2-(2-methoxyphenyl)acetamide;
N-[3-(2-amino-4-cyclohexyl-1-methyl-5-oxo-4,5-dihydro-1H-imidazol-4-yl)phenyl]-1-methyl-1H-pyrrole-2-carboxamide;
N-[3-(2-amino-4-cyclohexyl-1-methyl-5-oxo-4,5-dihydro-1H-imidazol-4-yl)phenyl]-2-methoxyacetamide;
N-[3-(2-amino-4-cyclohexyl-1-methyl-5-oxo-4,5-dihydro-1H-imidazol-4-yl)phenyl]-2-furamide;
N-[3-(2-amino-4-cyclohexyl-1-methyl-5-oxo-4,5-dihydro-1H-imidazol-4-yl)phenyl]-2-(benzyloxy)acetamide;
N-[3-(2-amino-4-cyclohexyl-1-methyl-5-oxo-4,5-dihydro-1H-imidazol-4-yl)phenyl]-2-methoxybenzamide;
N-[3-(2-amino-4-cyclohexyl-1-methyl-5-oxo-4,5-dihydro-1H-imidazol-4-yl)phenyl]-3,4-dimethoxybenzamide;
N-[3-(2-amino-4-cyclohexyl-1-methyl-5-oxo-4,5-dihydro-1H-imidazol-4-yl)phenyl]-2-(2,5-dimethoxyphenyl)acetamide;
(2E)-N-[3-(2-amino-4-cyclohexyl-1-methyl-5-oxo-4,5-dihydro-1H-imidazol-4-yl)phenyl]but-2-enamide;
N-[3-(2-amino-4-cyclohexyl-1-methyl-5-oxo-4,5-dihydro-1H-imidazol-4-yl)phenyl]butanamide;
N-[3-(2-amino-4-cyclohexyl-1-methyl-5-oxo-4,5-dihydro-1H-imidazol-4-yl)phenyl]-2-(3-methoxyphenyl)acetamide;
N-[3-(2-amino-4-cyclohexyl-1-methyl-5-oxo-4,5-dihydro-1H-imidazol-4-yl)phenyl]-1,3-benzodioxole-5-carboxamide;
N-[3-(2-amino-4-cyclohexyl-1-methyl-5-oxo-4,5-dihydro-1H-imidazol-4-yl)phenyl]-2-(4-chlorophenoxy)-2-methylpropanamide;
or a tautomer thereof or a stereoisomer thereof or a pharmaceutically acceptable salt thereof.
